(19) United States
(12) Patent Application Publication
(10) Pub. No.: US 2026/0335336 A1
(43) Pub. Date: Oct. 29, 2026

(54) PRECISION MEDICINE FOR ANXIETY DISORDERS: OBJECTIVE ASSESSMENT, RISK PREDICTION, PHARMACOGENOMICS, AND REPURPOSED DRUGS

(71) Applicants: Indiana University Research and Technology Corporation, Bloomington, IN (US); THE UNITED STATES GOVERNMENT AS REPRESENTED BY THE DEPARTMENT OF VETERANS AFFAIRS, Washington D.C., DC (US)

(72) Inventor: Alexander Bogdan Niculescu, Indianapolis, IN (US)

(21) Appl. No.: 19/099,215

(22) PCT Filed: Jul. 28, 2022
(86) PCT No.: PCT/US2022/038673

§ 371 Date: Jan. 28, 2025

(51) Int. Cl. A61K 31/565; A61K 31/135; A61K 31/165; A61K 31/19; A61K 31/343; A61K 31/451; A61K 31/495; A61K 31/496; A61K 31/55; A61K 31/5513; G16H 20/10; G16H 50/30

(52) U.S. Cl. CPC A61K 31/565; A61K 31/135; A61K 31/165; A61K 31/19; A61K 31/343; A61K 31/451; A61K 31/495; A61K 31/496; A61K 31/55; A61K 31/5513; G16H 20/10; G16H 50/30

(57) ABSTRACT

We used a powerful longitudinal within-subject design in individuals with psychiatric disorders to discover blood gene expression change between self-reported low anxiety and high anxiety states. We prioritized the list of candidate biomarkers with a Convergent Functional Genomics approach using other evidence in the field and validated our top biomarkers from discovery and prioritization in an independent cohort of psychiatric subjects with clinically severe anxiety. We then tested if these candidate biomarkers are able to predict anxiety severity state, and future clinical worsening (hospitalizations with anxiety as a contributory cause), in another independent cohort of psychiatric subjects. We showed increased accuracy of individual biomarkers with a personalized approach, by gender and diagnosis, particularly in women. Finally, we identified which of our biomarkers are targets of existing drugs and thus can be used for pharmacogenomic population stratification and measuring of response to treatment, as well as used biomarker gene expression signatures to identify drugs that can be repurposed for treating anxiety.

### STATEMENT OF GOVERNMENT SUPPORT

This invention was made with government support under MH117431 awarded by the National Institutes of Health and CX000139 merit award by the Veterans Administration. The government has certain rights in the invention.

### BACKGROUND

Anxiety disorders are highly prevalent and insidious in their effects on one's ability to do things and quality of life. Psychiatric patients may have increased anxiety, as well as increased reasons for anxiety, due to their often, adverse life trajectory. Improvements are needed to adequately diagnose and treat all individuals suffering with anxiety disorders.

### SUMMARY

Provided here are newly identified blood gene expression biomarkers for anxiety. The biomarkers provide a means of assessing state severity, short-term risk, and long-term risk. The biomarkers can also be used for drug repurposing.

Some aspects of the invention include methods for treating an individual experiencing or at a heighted risk for developing symptoms is anxiety disorders. Treating involves administered at least one course of treatment, treatment may include psychiatric counseling, administering certain physical intervention, and/or prescribing and/or administering at least one therapeutic compound. Treating may include at least one of the following outcomes, curing, mitigating, managing or otherwise recuing the severity and/or the number of frequency anxious behaviors, especially behaviors that negatively impact a individual's quality of life.

A first embodiment is a therapeutic compound selected from one or more compounds in Tables 4 and Table 6, for use in a method of mitigating and anxiety disorder in a subject in need thereof for example wherein the therapy is a drug, a natural compound, and combinations thereof.

A second embodiment is the therapeutic compound for use according to the first embodiment, wherein the drug is selected from the group consisting of: estradiol, loperamide, omage-3 fatty acids, lithium, fluoxetine, norfluoxetine, imipramine, citalopram, carbamazepine, aripiprazole, haloperidol, mianserin, SAM, vortioxetine, agomelatine, sertraline, benzodiazepine, clozapine, ketamine, valproate, clozapine, rofecoxib, berberine, methylphenidate, or berberine.

A third embodiment is the therapeutic compound for use according to the first embodiment, wherein the subject has an anxiety disorder.

A fourth embodiment is a therapeutic compound for use according to the first embodiment, wherein the subject is a male subject or a female subject.

A fifth embodiment is a computer-implemented method for assessing the level of anxiety in a subject, and for assessing risk of developing anxiety disorder in the future in a subject, the method comprising: computing a score based on RNA level, protein level, DNA methylation, a single nucleotide polymorphism, for a panel of biomarkers chosen from Table 2, Table 5, and FIG. 2, in a sample obtained from a subject; computing a score based on a reference expression level of the panel of biomarkers; and identifying a difference between the score in the sample obtained from the subject and the score in the reference sample, wherein the difference in the score in the sample obtained from the subject and the score in the reference sample indicates an anxiety disorder, and a risk of future anxiety disorders.

A sixth embodiment is the method according to the fifth embodiment, wherein the subject is: a male or a female and the biomarker is at least one biomarker selected from the group consisting of: ERCC6L2, SLC6A2, FZD10, and RPL14; or a female and the biomarker is at least one biomarker selected from the group consisting of: PHF21A, SLC6A2, 240253_at, and TRIM9; or a male and the biomarker is at least one biomarker selected from the group consisting of: ERCC6L2, ANKRD28, ACP1, and RP

A seventh embodiment is the method according to the fifth or the sixth embodiments, further including the steps of: identifying a difference between the score in the sample obtained from the subject and the score in the reference sample, administering treatment to the subject, wherein the treatment reduces the difference between the score in the sample from the subject and the score in the reference sample, and mitigates anxiety in the subject, or the risk for developing anxiety disorder in the subject.

An eighth embodiment is the method according to the seventh embodiment, further including, the step of: measuring a change in the score after treatment.

A ninth embodiment is the method according to the eighth embodiment, wherein the treatment is at least one treatment selected from the group consisting of: lifestyle modification, electro-magnetic intervention, and administering a therapeutic compound.

A tenth embodiment is the method according to the ninth embodiment, wherein the therapy is selected by a computer-implemented method selected from the group consisting of one or more psychiatric compounds from Table 6, and wherein each therapy selection is based on a panel of one or more individual biomarkers.

An eleventh embodiment is the method according to the tenth embodiment, wherein the therapy is selected based on a panel of individual biomarkers changed in a subject, by a computer-implemented method for therapy selection, and comprises, of one or more compounds in Table 4.

An twelfth embodiment is a method for assessing and mitigating anxiety disorder, comprising determining an expression level of a panel of biomarkers listed in Table 2, Table 5 and FIG. 2, in a sample for example wherein the sample comprises a peripheral tissue, blood, saliva, cerebrospinal fluid (CSF), serum, urine, or stool; wherein the expression level of the biomarkers in the sample is different relative to a reference expression level; and includes the steps of: identifying the subject currently having or at risk of having an anxiety disorder based on a biomarker panel score relative to a biomarker panel score of a reference; selecting a therapy based on the score from the biomarkers, wherein the therapy includes; and administering one or more compounds selected from the group consisting of compounds selected from the groups consisting of compounds selected from Tables 4 and Table 6.

A thirteenth embodiment is a composition comprising one or more compounds selected from Tables 4 and Table 6, for use in a method for treating anxiety disorder.

### BRIEF DESCRIPTION OF THE FIGURES

FIG. 1A. Discovery cohort, within subject changes in anxiety state (N of 58).

FIG. 1 B. Discovery cohort 141 male and 17 female psychiatric participants have at least one switch between a low anxiety state visit and a high anxiety state visit.

FIG. 1 C. Convergent functional genomics. Multiple independent lines of evidence for identification and prioritization of anxiety biomarkers.

FIG. 1 D. Graphic presentation of data. Left panel, EFNAS, P=2.02 E-04; right panel TG51, P=2.3E-03.

FIG. 1 E. Graphic representation of Discovery, Prioritization and Validation of biomarkers.

FIG. 2A. Graph of predictions for state-high anxiety state (SAS4≥60). See also TABLE 6.

FIG. 2B. Graph of predictions for state-high STAI (STA≥55). Light colored bars, cross sectional; dark colored bars, longitudinal.

FIG. 2C. Graph of anxiety trait predictions first year. Light colored bars, cross sectional; dark colored bars, longitudinal.

FIG. 2D. Graph of anxiety trait predictions-all future year. Light colored bars, cross sectional; dark colored bars, longitudinal.

FIG. 3. String network analysis. For top candidate biomarkers (n=82 genes, 95 probesets).

FIG. 4. Summary of an analysis of Phchp328, a specific Female subject, MDD, 37 years old, Caucasian; SAS4=92.5; STAI State=68/80. Anxiety Score 86.67% (13/15) (High), Chronic Anxiety Risk: 88.89% (High).

FIG. 5. SASA4 scaler for measuring anxiety State (Niculescu, et al., 006, 2015). The score is an average of 4 items, Ranges from 0-100.

FIG. 6. Graphical depiction of the correlation between SAS4 and STAI State. Our SAS4 scale shows a moderate to strong correlation with a well-validated clinical measure of anxiety, the STAI State, while being more temporally related to that particular moment in time, quantitative, and simple. Pearson Correlation Coefficient 0.667503766; sample size 784; test statistic 25.06861378; p-value 1.4989E-102.

### DETAILED DESCRIPTION

We endeavored to find objective blood biomarkers for anxiety disorders. First, we used a powerful longitudinal within-subject design in individuals with psychiatric disorders to discover blood gene expression changes between self-reported low anxiety and high anxiety states, as measured by a visual analog scale we developed, the Simplified Anxiety Scale (SAS-4). Second, we prioritized the list of candidate biomarkers with a Bayesian-like Convergent Functional Genomics approach, comprehensively integrating previous human and animal model evidence in the field. Third, we validated our top biomarkers from discovery and prioritization in an independent cohort of psychiatric subjects with clinically severe anxiety. We prioritized a list of 95 candidate biomarkers that had the most evidence from the first three steps. Fourth, we tested if these candidate biomarkers are able to predict anxiety severity state, and future clinical worsening (hospitalizations with anxiety as the primary cause), in another independent cohort of subjects. We tested the biomarkers in all subjects in the test cohort, as well as in a more personalized fashion by gender and anxiety disorder diagnosis, showing increased accuracy with the personalized approach, particularly in women. Fifth, we analyzed the biological pathways and networks the biomarkers are involved in, as well as which of our top biomarkers have evidence for involvement in other psychiatric and related disorders. Sixth, we identified which of our biomarkers are targets of existing drugs and thus can be used for pharmacogenomic population stratification and measuring of response to treatment, as well as used the top biomarkers gene expression signature to identify drugs and natural compounds that can be repurposed for treating anxiety.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is generally directed at methods for assessing anxiety disorders and early identification of risk for future episodes of acute or chronic anxiety, as well as methods for matching patients and drugs for prevention and mitigation of anxiety and for monitoring response to treatment. The methods may further include the generation of a report providing a risk score and/or personalized treatment options. Further, the present disclosure generally is directed to drugs for mitigating anxiety in subjects. Particular drugs have been found that can mitigate anxiety in subjects universally; that is, drugs that can be used for mitigating anxiety across genders an in the presence of confounding factors such as other psychiatric disorders. Some drugs, however, have been found that can be used more effectively for mitigating anxiety dependent on gender, confounding factors, and combinations thereof.

In additional embodiments, the present disclosure is directed to blood gene expression biomarkers that are more universal in nature; that is, blood biomarkers that can be used for predicting anxiety disorders in one of more genders. Accordingly, a longitudinal within-participant design and large cohorts were used. Additionally, subtypes of anxiety were identified based on mental state at the time that anxiety was manifest.

### Materials and Methods

### Cohorts

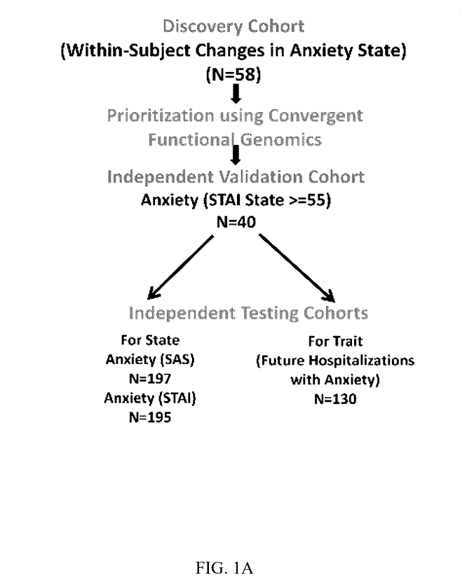

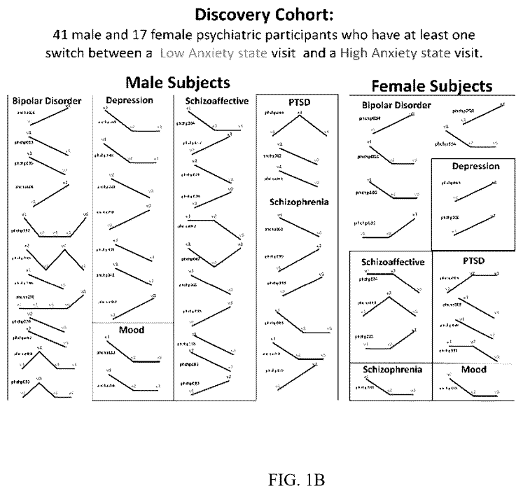

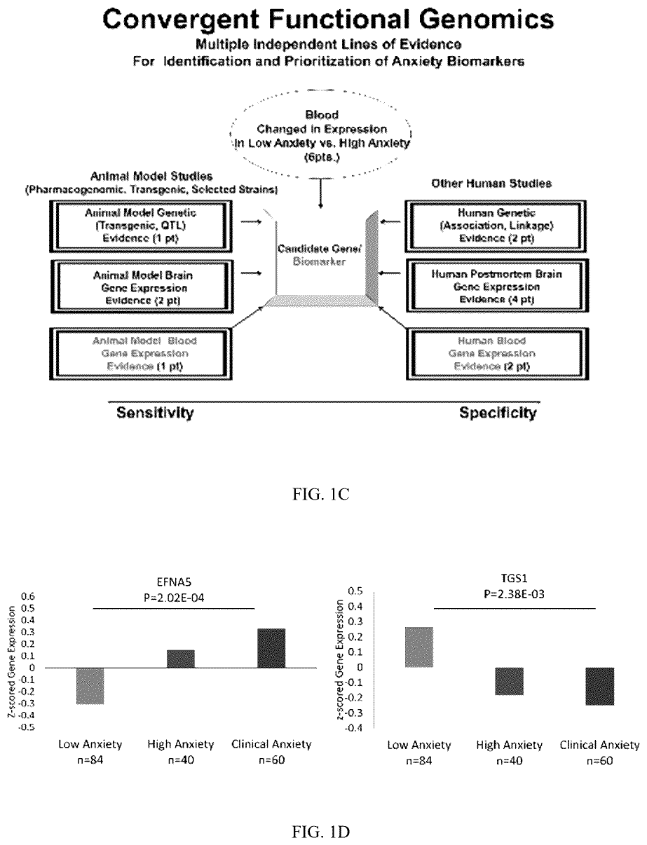

We used three independent cohorts: discovery (major psychiatric disorders with changes in state anxiety), validation (major psychiatric disorders with clinically severe anxiety), and testing (an independent major psychiatric disorders cohort for predicting state anxiety, and for predicting trait anxiety (future hospitalization with anxiety as the primary reason) (FIG. 1).

Referring now to FIG. 1. FIG. 1. Steps 1-3: Discovery, Prioritization and Validation of Biomarkers for Anxiety (A.) Cohorts used in study, depicting flow of discovery, prioritization, validation of biomarkers from each step and independent testing cohorts. (B.) Discovery cohort longitudinal within-subject analysis. Phchp ### is study ID for each subject. V # denotes visit number. C.) Convergent Functional Genomics lines of evidence from human to non-human. (D) In the validation step biomarkers are assessed for stepwise change from the validation group with severe Anxiety, to the discovery groups of subjects with high Anxiety, low Anxiety, to the validation group with severe Anxiety, using ANOVA. N-number of testing visits. The histograms depict a top increase and a top decrease Validator biomarkers (E) Pyramid schemes and scoring at each of the steps. Discovery probesets are identified based on their score for tracking Mood with a maximum of internal points of 6 (33% (2 pt), 50% (4 pt) and 80% (6 pt)). Prioritization with CFG for prior evidence of involvement in mood disorders. In the prioritization step probesets are converted to their associated genes using Affymetrix annotation and GeneCards. Genes are prioritized and scored using CFG for Anxiety evidence with a maximum of 12 external points. Genes scoring at least 6 points out of a maximum possible of 18 total internal and external scores points are carried to the validation step. Validation in an independent cohort of psychiatric patients with clinically severe Anxiety (STAI State≥55 and SAS4>=60). 4 biomarkers were nominally significant, and 57 biomarkers were stepwise changed. We selected the top CFE3 score≥8 (n=95) for further testing in independent cohorts.

Similar to our previous studies, the psychiatric subjects are part of a larger longitudinal cohort of adults that we are continuously collecting. Subjects were recruited from the patient population at the Indianapolis VA Medical Center. All subjects understood and signed informed consent forms detailing the research goals, procedure, caveats and safeguards, per IRB approved protocol. Subjects completed diagnostic assessments and extensive structured neuropsychological testing at each testing visit, 3-6 months apart or whenever a new psychiatric hospitalization occurred. At each testing visit, they received a series of rating scales, including a self-report visual analog scale (1-100) for quantitatively assessing state anxiety at that particular moment in time (Simplified Anxiety Scale-SAS-4). This 4-item scale looks at anxiety overall, as well as fear, anger, and uncertainty. Each of the items are a VAS of 0 to 100, related to that moment in time. As such, it generates temporal, quantitative, and targeted data.

At each testing visit we collected whole blood (10 ml) in two RNA-stabilizing PAXgene tubes, labeled with an anonymized ID number, and stored at −80 degrees C in a locked freezer until the time of future processing. Whole-blood RNA was extracted for microarray gene expression studies from the PAXgene tubes, as detailed below.

For this study, our within-subject discovery cohort, from which the biomarker data were derived, consisted of 58 subjects (41 males, 17 females) with multiple testing visits, who each had at least one diametric change in anxiety state from low anxiety state (SAS-4 score of ≤40/100) to a high anxiety state (SAS-4 score of ≥60/100), or vice versa, from one testing visit to another (FIG. 1). There were 2 subjects with 5 visits each, 3 subjects with 4 visits each, 21 subjects with 3 visits each, and 32 subjects with 2 visits each resulting in a total of 149 blood samples for subsequent gene expression microarray studies (FIG. 1, Table 1).

Our independent validation cohort, in which the top biomarker findings were validated for being even more changed in expression, consisted of 40 subjects (32 male and 8 female) with clinically severe anxiety (SAS-4 scores≥60, and concordant high anxiety STAI State scores≥55 (Table 1).

We use independent test cohorts for predicting high anxiety state (SAS-4≤60) consisted of 161 male and 36 female subjects and another test cohorts, using (STAI≥55) consisted of 159 male and 36 female subjects with psychiatric disorders, demographically matched with the discovery cohort, with one or multiple testing visit in our study, with either low anxiety, intermediate anxiety, or high anxiety states based on the two state scales (FIG. 1 and Table 1).

For trait predictions of future hospitalizations with anxiety as the primary reason (FIG. 1 and Table 1), are a subset of the independent test cohort for which we had longitudinal follow-up with electronic medical records. The subjects' subsequent number of hospitalizations with anxiety was tabulated from electronic medical records.

### Medications.

The subjects in the discovery cohort were all diagnosed with various psychiatric disorders (Table 1) and had various medical co-morbidities. Their medications were listed in their electronic medical records and documented by us at the time of each testing visit. Medications can have a strong influence on gene expression. However, there was no consistent pattern of any particular type of medication, as our subjects were on a wide variety of different medications, psychiatric and non-psychiatric. Furthermore, the independent validation and testing cohort's gene expression data was Z-scored by gender and by diagnosis before being combined, to normalize for any such effects. Some subjects may be non-compliant with their treatment and may thus have changes in medications or drug of abuse not reflected in their medical records. That being said, our goal is to find biomarkers that track anxiety, regardless if the reason for it is endogenous biology or it is driven by medications or drugs. In fact, one would expect some of these biomarkers to be targets of medications, as we show in this paper. Moreover, the prioritization step that occurs after discovery is based on a field-wide convergence with literature that includes genetic data and animal model data, that are unrelated to medication effects. Overall, the discovery, validation and replication by testing in independent cohorts of the biomarkers, with our design, occurs despite the subjects having different genders, diagnoses, being on various different medications, and other lifestyle variables.

### Blood Gene Expression Experiments

RNA extraction. Whole blood (2.5 ml) was collected into each PaxGene tube by routine venipuncture. PaxGene tubes contain proprietary reagents for the stabilization of RNA. Total RNA was extracted and processed as previously described.

Microarrays. Microarray Work was Carried Out Using Previously Described Methodology¹.

Of note, all genomic data was normalized (RMA for technical variability, then z-scoring for biological variability), by gender and psychiatric diagnosis, before being combined and analyzed.

### Biomarkers

### Step 1: Discovery

We have used the subject's score from a visual-analog scale (SAS-4) scale, assessed at the time of blood collection (FIG. 1). We analyzed gene expression differences between visits with low anxiety (defined as a score of 0-40) and visits with high anxiety (defined as a score of 60-100), using a powerful within-subject design, then an across-subjects summation (FIG. 1).

We analyzed the data in two ways: an Absent-Present (AP) approach, and a differential expression (DE) approach, as in previous work by us on suicide biomarkers. The AP approach may capture turning on and off of genes, and the DE approach may capture gradual changes in expression. Analyses were performed as previously described²⁻⁴. In brief, we imported all Affymetrix microarray data as CEL. files into Partek Genomic Suites 6.6 software package (Partek Incorporated, St Louis, MI, USA). Using only the perfect match values, we ran a robust multi-array analysis (RMA) by gender and diagnosis, background corrected with quantile normalization and a median polish probeset summarization of all chips, to obtain the normalized expression levels of all probesets for each chip. Then, to establish a list of differentially expressed probesets we conducted a within-subject analysis, using a fold change in expression of at least 1.2 between consecutive high- and low anxiety visits within each subject. Probesets that have a 1.2-fold change are then assigned either a 1 (increased in high anxiety) or a −1 (decreased in high anxiety) in each comparison. Fold changes between 1.1 and 1.2 are given 0.5, and fold changes less than 1.1 are given 0. These values were then summed for each probeset across all the comparisons and subjects, yielding a range of raw scores. The probesets above the 33.3% of raw scores were carried forward in analyses (FIG. 1), and received an internal score of 2 points; those above 50% 4 points, and those above 80% 6 points^{2 3 4}. We have developed in our labs R scripts to automate and conduct all these large dataset analyses in bulk, checked against human manual scoring⁴.

Gene Symbol for the probesets were identified using NetAffyx (Affymetrix) for Affymetrix HG-U133 Plus 2.0 GeneChips, followed by GeneCards to confirm the primary gene symbol. In addition, for those probesets that were not assigned a gene symbol by NetAffyx, we used GeneAnnot, or if need be UCSC to obtain gene symbol for these uncharacterized probesets, followed by GeneCard. Genes were then scored using our manually curated CFG databases as described below (FIG. 1C).

### Step 2: Prioritization Using Convergent Functional Genomics (CFG)

### Databases.

We have established in our laboratory (Laboratory of Neurophenomics) manually curated databases of the human gene expression/protein expression studies (postmortem brain, peripheral tissue/fluids: CSF, blood and cell cultures), human genetic studies (association, copy number variations and linkage), and animal model gene expression and genetic studies, published to date on psychiatric disorders. Only findings deemed significant in the primary publication, by the study authors, using their particular experimental design and thresholds, are included in our databases. Our databases include only primary literature data and do not include review papers or other secondary data integration analyses to avoid redundancy and circularity. We also favored unbiased discovery studies over candidate genes hypothesis-driven studies. These large and constantly updated databases have been used in our CFG cross validation and prioritization platform (FIG. 1E). For this study, data from 354 papers on anxiety were present in the databases at the time of the CFG analyses (July 2019) (human genetic studies-93, human brain studies-10, human peripheral tissue/fluids-96, non-human genetic studies-17, non-human brain studies-123, non-human peripheral tissue/fluids-17). Analyses were performed as previously described. We have developed in our lab a computerized CFG Wizard to automate and score in bulk large lists of genes by integrating evidence from these large databases, checked against manual scoring. Analyses were performed as previously described^{2, 3}.

### Step 3: Validation Analyses

We examined which of the top candidate genes (score of 6 or above after the first two steps), were changed in expression even more in an independent validation cohort (n=40) of clinically severe anxiety as measured by a STAI State≥55, as well as a SAS-4≥60. A total score of 6 or above after the first two steps permits the inclusion of potentially novel genes with maximal internal score of 6 from Discovery but no external evidence CFG score from Prioritization.

Subjects with low anxiety as well as subjects with high anxiety from the discovery cohort who did not have clinically severe anxiety were used, along with the independent validation cohort. We looked for stepwise change from the discovery cohort low anxiety group to the discovery cohort high anxiety group to the validation cohort clinically severe group.

The AP derived and DE derived lists of genes were combined, and the gene expression data corresponding to them was used for the validation analysis. The 3 groups (low anxiety, high anxiety, clinical severe anxiety) were assembled out of Affymetrix. cell data that was RMA normalized by gender and diagnosis. We transferred the log transformed expression data to an Excel sheet, and non-log transformed the data by taking 2 to the power of the transformed expression value. We then Z-scored the values by gender and diagnosis. We then imported the Excel sheets with the Z-scored by gender and diagnosis expression data into Partek, and statistical analyses were performed using a one-way ANOVA for the stepwise changed probesets, and also did a stringent Bonferroni correction for all the probesets tested in ANOVA (FIG. 1).

Top Candidate Biomarkers (after the First 3 Steps)

Adding the scores from the first three steps into an overall convergent functional evidence (CFE) score (FIG. 1), we ended up with a list of 95 top candidate biomarkers (95 probesets in n=82 genes), that had a CFE score of 8 and above (out of a maximum possible of 24; ≥33%) (see also Supplementary Information-Pathways, Predictions and Reproducibility). These 95 top candidate biomarkers were carried forward into additional analyses for biological understanding, and for testing for clinical utility (Step 4).

### Biological Understanding

### Pathway Analyses

IPA (Ingenuity Pathway Analysis, version 24390178, Qiagen), David Functional Annotation Bioinformatics Microarray Analysis (National Institute of Allergy and Infectious Diseases) version 6.7 (August 2016), and Kyoto Encyclopedia of Genes and Genomes (KEGG) (through DAVID) were used to analyze the biological roles, including top canonical pathways and diseases (Table 3).

### Networks

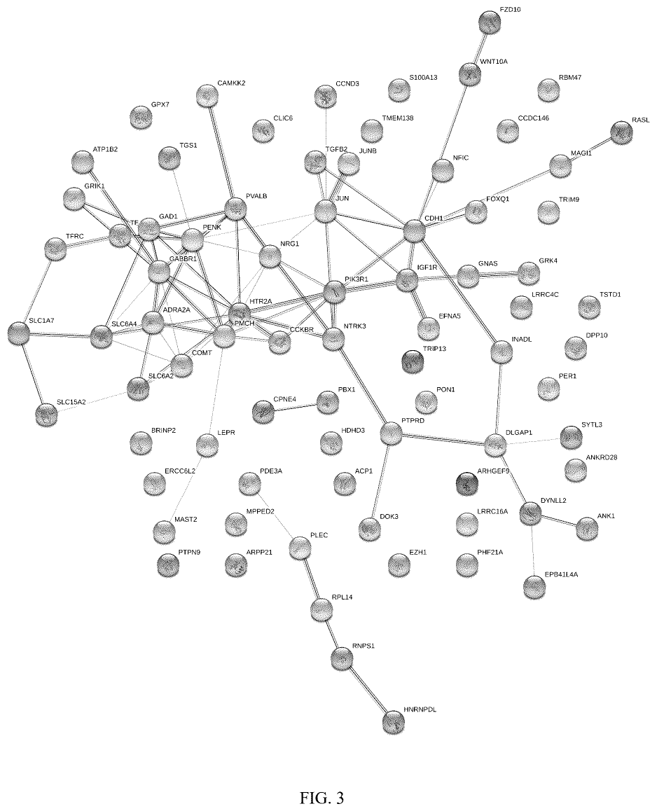

For network analyses we performed STRING Interaction network (https://string-db.org) by inputting the genes into the search window and performed Multiple Proteins Homo sapiens analysis (FIG. 3).

### CFG Beyond Anxiety: Evidence for Involvement in Other Psychiatric and Related Disorders.

We also used a CFG approach to examine evidence from other psychiatric and related disorders, as exemplified for the list of top biomarkers after Step 4 testing (Table 2). This was not used to prioritize genes, but rather to understand the molecular basis of clinical co-morbidities.

### Testing for Clinical Utility in Independent Cohorts

We tested in independent cohorts of psychiatric patients the ability of each of the top candidate biomarkers (n=95) to assess current severity of anxiety (state-measured by SAS-4 or STAI), and predict future risk of anxiety (trait-future hospitalizations with anxiety). We conducted our analyses across all patients, as well as personalized by gender and diagnosis.

The test cohort for predicting high anxiety (state), and the test cohort for predicting future hospitalizations with anxiety (trait), were assembled out of data that was RMA normalized by gender and diagnosis. The cohort was completely independent from the discovery and validation cohorts, there was no subject overlap with them. Individual markers used for predictions were Z scored by gender and diagnosis, to be able to combine different biomarkers into panels and to avoid potential artefacts due to different ranges of expression in different gender and diagnoses. For panels, biomarkers were combined by simple summation of the increased risk biomarkers minus the decreased risk biomarkers. Predictions were performed using R-studio. For cross-sectional analyses, we used biomarker expression levels, z-scored by gender and diagnosis. For longitudinal analyses, we combined four measures: biomarker expression levels, slope (defined as ratio of levels at current testing visit vs. previous visit, divided by time between visits), maximum levels (at any of the current or past visits), and maximum slope (between any adjacent current or past visits). For decreased biomarkers, we used the minimum rather than the maximum for level calculations. All four measures were Z-scored, then combined in an additive fashion into a single measure. The longitudinal analysis was carried out in a sub-cohort of the testing cohort consisting of subjects that had at least two visits (timepoints).

### Predicting State-High Anxiety.

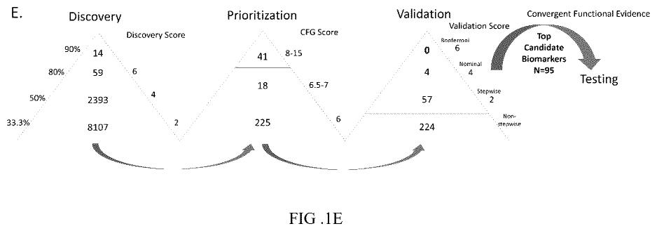

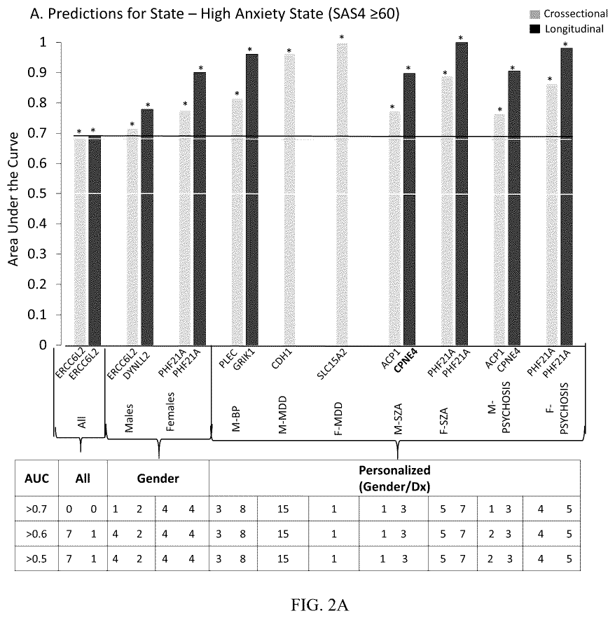

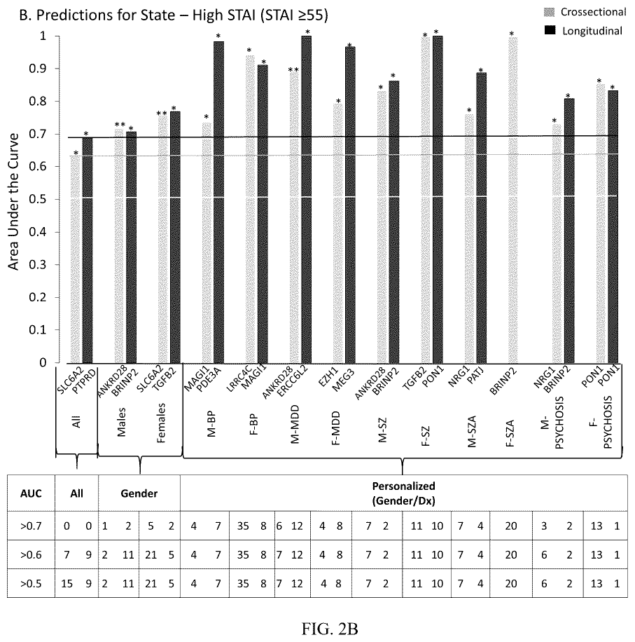

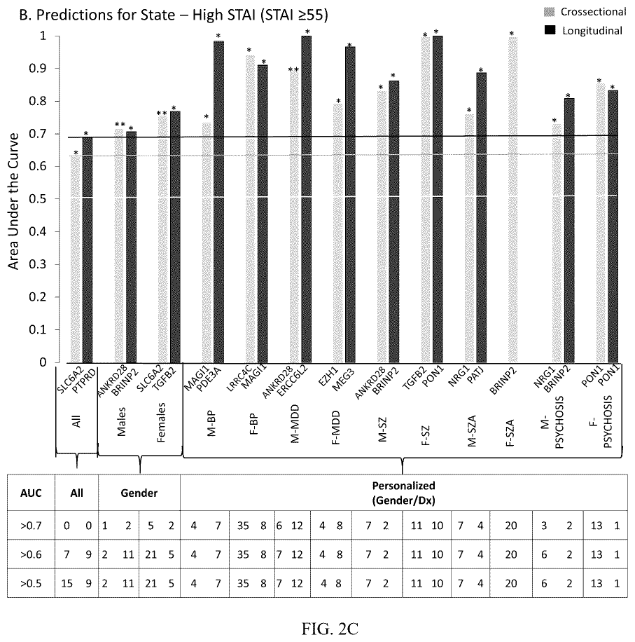

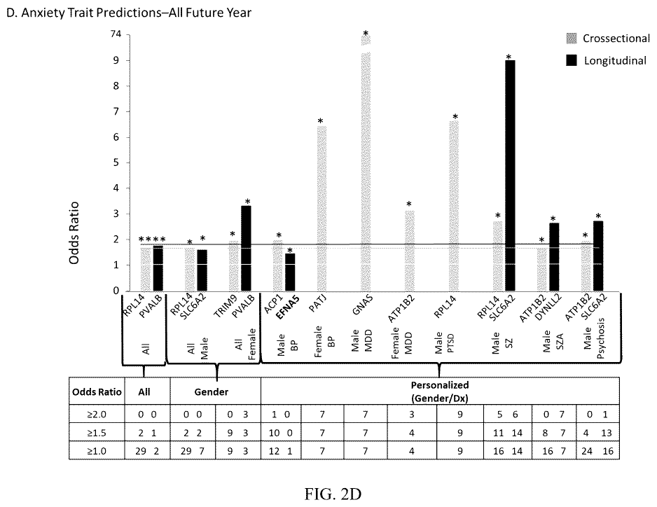

Receiver-operating characteristic (ROC) analyses between marker levels and anxiety state were performed by assigning subjects visits with a anxiety SAS-4 score of ≥60 into the high anxiety category, and subjects with STAI scores≥55 in the high anxiety category. We used the pROC package of R (Xavier Robin et al. BMC Bioinformatics 2011). (Table 3, FIG. 2). Additionally, a one-tailed t-test was performed between high anxiety group vs. the rest, and Pearson R (one-tail) was calculated between anxiety scores and biomarker levels.

Referring now to FIG. 2. FIG. 2, Best Single Biomarkers Predictors for Anxiety State and Trait. From top candidate biomarkers after Steps 1-3 (Discovery, Prioritization, Validation-Bold) (n=95). Bar graph shows best predictive biomarkers in each group. All markers with * are nominally significant p<0.05. Table underneath the figures displays the actual number of biomarkers for each group whose ROC AUC p-values (2 A-C) and Cox Odds Ratio p-values (2 D) are at least nominally significant. Some gender and diagnosis group are missing from the graph as they did not have any significant biomarkers or that the cohort was too small with limited data for the z-scoring by gender-dx. Cross-sectional is based on levels at one visit. Longitudinal is based on levels at multiple visits (integrates levels at most recent visit, maximum levels, slope into most recent visit, and maximum slope). Dividing lines represent the cutoffs for a test performing at chance levels (white), and at the same level as the best biomarkers for all subjects in cross-sectional (gray) and longitudinal (black) based predictions. Biomarkers perform better than chance. Biomarkers performed better when personalized by gender and diagnosis. * survived Bonferroni correction for the number of candidate biomarkers tested.

Predicting Trait-Future Psychiatric Hospitalization with Anxiety as a Symptom/Reason for Admission.

We conducted analyses for predicting future psychiatric hospitalizations with anxiety as a symptom/reason for the visit or admission in the first year following each testing visit, in subjects that had at least one year of follow-up in the VA system, in which we have access to complete electronic medical records. ROC analyses between biomarkers measures (cross-sectional, longitudinal) at a specific testing visit and future hospitalization admission were performed as described above, based on assigning if subjects had been admitted to the hospital with depression or not. Additionally, a one tailed t-test with unequal variance was performed between groups of subject visits with and without future hospitalization with anxiety. Pearson R (one-tail) correlation was performed between Hospitalization frequency (number of Hospitalization with anxiety divided by duration of follow-up) and marker levels. A Cox regression was performed using the time in days from the testing visit date to first hospitalization date in the case of patients who had been hospitalized, or 365 days for those who did not. The odds ratio was calculated such that a value greater than 1 always indicates increased risk for hospitalization, regardless if the biomarker is increased or decreased in expression.

We also conducted Cox regression and Pearson R analyses for all future hospitalizations with anxiety, including those occurring beyond one year of follow-up, in the years following testing (on average 7.35 years per subject, range 0.07 to 14.74 years), as these calculations, unlike the ROC and t-test, account for the actual length of follow-up, which varied from subject to subject. The ROC and t-test might in fact, if used, under-represent the power of the markers to predict, as the more severe psychiatric patients are more likely to move geographically and/or be lost to follow-up. The Cox regression was performed using the time in days from visit date to first hospitalization date in the case of patients who had hospitalizations with anxiety, or from visit date to last note date in the electronic medical records for those who did not.

### Therapeutics

### Pharmacogenomics.

We analyzed which of the top biomarkers for anxiety after Steps 1~4 are known to be changed in expression by existing drugs in a direction opposite to the one in disease, using our CFG databases.

### New Drug Discovery/Repurposing

We also analyzed which drugs and natural compounds are an opposite match for the gene expression signatures of our top biomarkers, using the Connectivity Map (Broad Institute, MIT) (Table 4). Of note, not all the probesets from the HG-U133 Plus 2.0 array we used were present in the HGU-133A array used for the Connectivity Map. We stayed with exact probeset level matches, not gene level imputation. We also used the NIH LINCS tools database L1000CDS² https://maayanlab.cloud/L1000CDS2/#/index to conduct similar analyses, at a gene level.

### Report Generation

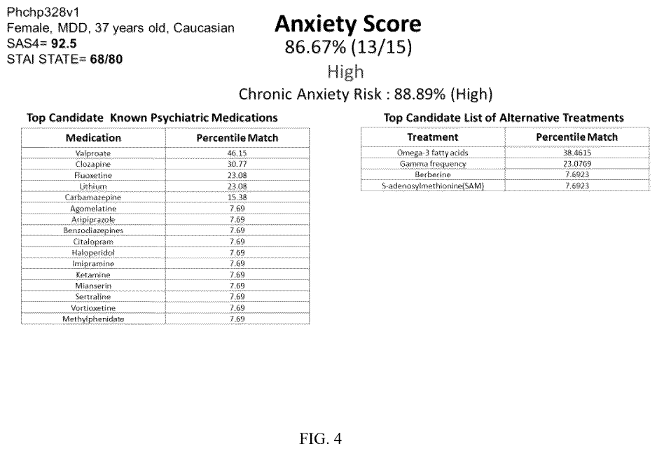

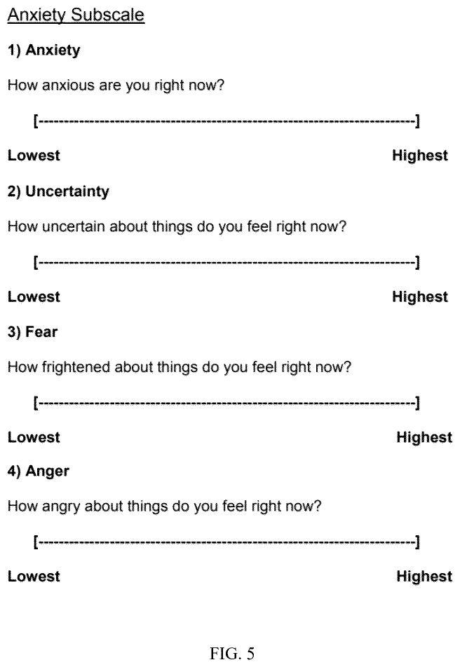

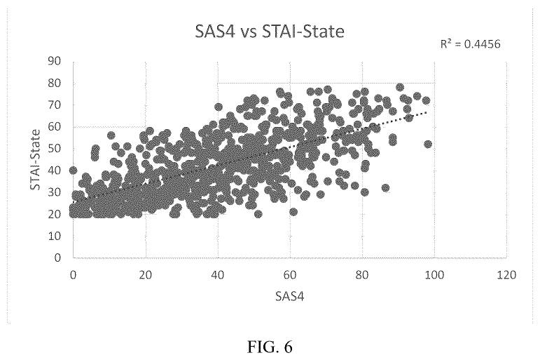

We present an example of how a report to doctors might look, using the above insights. We chose as a case study a visit from a female subject (phchp328v1) with anxiety and depression who had died by suicide, a case previously discussed in a suicide biomarker paper of ours (Levey et al. 2016³) (FIG. 4). We used the panel of the top biomarkers for anxiety from Table 2 (n=19).

### Example of Report for Physicians

Referring now to FIG. 4: Example of report for physicians. Using the panel of the top biomarkers for anxiety from Table 2 (n=19). This subject (Phchp328) was previously described by us in a suicidality biomarker study (Levey et al. 2016), as high risk for suicide, and died by suicide a year after completing our study. No information was provided to the patient's clinicians by us at that time due to anonymity and privacy rules in research studies.

The raw expression values of the 19 biomarkers for 794 microarrays gene expression were Z-scored by gender and diagnosis. We calculated as thresholds the average expression value for a biomarker in the high anxiety group SAS4≥60, and in the low anxiety group SAS4≤40. The first average should be higher than the second average in increased biomarkers, and the reverse is true for decreased biomarkers. 15 out of 19 biomarkers were thus concordant.

We also calculated as thresholds the average expression value for a biomarker in the first-year hospitalizations group, and in the not hospitalized in the first-year group. We did the same thing for all future hospitalizations, and no future hospitalizations. The first average should be higher than the second average in increased biomarkers, and the reverse is true for decreased biomarkers. 18 out of 19 biomarkers were thus concordant for first year, and for all future.

The Z-scored expression value of each increased in expression biomarker was compared to the average value for the biomarker in the high anxiety group SAS4≥60, and the average value of the low anxiety group SAS4≤40, resulting in scores of 1 if above high anxiety, 0 if below low anxiety, and 0.5 if it was in between. The reverse was done for decreased in expression biomarkers. This digitalization of the scores was done to avoid overfitting to our particular cohort and provide an easily understandable and interpretable readout for clinicians.

The digitized biomarkers were then added into a polygenic risk score and normalized for the number of biomarkers in the panel, resulting in a percentile score for anxiety. We did the same thing for first year hospitalizations, and all future hospitalizations, generating a combined score for chronic anxiety risk.

The digitized biomarkers were also used for matching with existing psychiatric medications and alternative treatments (nutraceuticals and others). We used our large datasets and literature databases to match biomarkers to medications that had effects on gene expression opposite to their expression in high anxiety. Each medication matched to a biomarker got the biomarker score of 1, 0.5 or 0. The scores for the medications were added, normalized for the number of biomarkers that were 1 or 0.5 in that patient, resulting in a percentile match. Thus, psychiatric medications matched to the patient and ranked in order of impact on the panel.

### Results

In Step 1 Discovery, we identified candidate blood gene expression biomarkers that: 1. change in expression in blood between self-reported low anxiety and high anxiety states, 2. track the anxiety state across visits in a subject, and 3. track the anxiety state in multiple subjects. We used a visual analog measure for anxiety state (SAS-4). At a phenotypic level, the SAS-4 quantitates anxiety state at a particular moment in time, and normalizes anxiety measurements in each subject, comparing them to the lowest and highest anxiety that subject ever experienced (Figure S1). It has a moderate to strong correlation with current clinical scales for anxiety state (the STAI State, Figure S2).

We used a powerful within-subject and then across-subject design in a longitudinally followed cohort of subjects (n=58 subjects, with 149 visits) who displayed at least a 50% change in the anxiety measure (from below 40/100 to above 60/100) between at least two consecutive testing visits, to identify differentially expressed genes that track anxiety state. Using our 33% of maximum raw score threshold (internal score of 2 pt)^{2,3}, we identified 10,573 unique probesets (corresponding to 7195 unique genes) from Affymetrix Absent/Present (AP) analyses and Differential Expression (DE) analyses (FIG. 1). These were carried forward to the prioritization step. This represents approximately a 5-fold enrichment of the 54,625 probesets on the Affymetrix array.

In Step 2 Prioritization, we used a Convergent Functional Genomics (CFG) approach to prioritize the candidate biomarkers identified in the discovery step (33% cutoff, internal score of ≥2 pt.) by using published literature evidence (genetic, gene expression and proteomic), from human and animal model studies, for involvement in anxiety disorders (FIG. 1 and Table 2). For Anxiety there were 284 probesets (corresponding to 238 unique genes) that had a total score (combined discovery score and prioritization CFG score) of 6 and above. These were carried forward to the validation step. This represents approximately a 10-fold enrichment of the probesets on the Affymetrix array.

In Step 3 Validation, we validated for change in clinically severe anxiety these prioritized biomarkers, in a demographically matched cohort of (n=40 clinically severe anxiety) by assessing which markers were stepwise changed in expression: from clinically severe anxiety in validation cohort, to high anixety in discovery cohort, to low anxiety in discovery cohort in the validation cohort (FIG. 1). 224 probesets were not stepwise changed, and 57 were stepwise changed. Of these, 4 probesets (corresponding to 4 unique genes) were nominally significant.

Adding the scores from the first three steps into an overall convergent functional evidence (CFE) score (FIG. 1), we ended up with a list of 95 top candidate biomarkers (n=82 genes, 95 probesets), that had a CFE3 score>=8, better than than 33% of the maximum possible score of 24 after the first three steps, which we decided to use as an empirical cutoff. This represents approximately an over 500-fold enrichment of the probesets on the Affymetrix array. These 95 top candidate biomarkers were carried forward into analyses for understanding biological underpinnings. Last but not least, they were tested for predictive ability and clinical utility in additional independent cohorts.

### Biological Understanding

### Biological Pathways.

We conducted biological pathway analyses using the two lists of top candidate biomarkers for anxiety (n=82 genes, 95 probesets), which suggest that CAMP signaling is involved (Table 3). Depression was a top disease identified by the pathway analyses using DAVID, pointing out to the issue of co-morbidity, and Ingenuity identified neurological disorders as the top diseases.

### Networks and Interactions.

We conducted a STRING analysis (FIG. 3) of the top candidate biomarkers that revealed groups of interacting proteins. In particular, HTR2A is at the overlap of a network containing GABBR1, GAD1 and SLC6A4, and one centered on CDH1 that also contains PIK3R1 and IGFR1. A third network includes DLGAP1, PTPRD and DYNLL2. These networks may have biological significance and could be targeted therapeutically.

Testing for Clinical Utility In Step 4 Testing, we examined in completely independent cohorts from the ones used for discovery or validation whether the 26 top candidate biomarkers can assess high anxiety state (n=190 subjects), as well as predict of future psychiatric hospitalizations with anxiety (n=170 subjects) (FIG. 2, and Table 2), using electronic medical records follow-up data of our study subjects (over a decade from initial visit at the time of the analyses) (FIG. 1, Table 1). The gene expression data in the test cohorts was normalized (Z-scored) across genders and various psychiatric diagnoses, before those different demographic groups were combined. We used biomarker levels information cross-sectionally, as well as expanded longitudinal information about biomarker levels at multiple visits, as predictors. We tested the biomarkers in all subjects in the independent test cohort, as well as in a more personalized fashion by gender and psychiatric diagnosis.

### Convergent Functional Evidence (CFE) (Table 2)

For the top candidate biomarkers (n=95), we tabulated into a convergent functional evidence (CFE4) score all the evidence from discovery (up to 6 points), CFG prioritization (up to 12 points), validation (up to 6 points), and testing (state high anxiety, state clinical anxiety, trait first year hospitalization with anxiety, trait all future hospitalizations with anxiety-up to 3 points each if it significantly predicts in all subjects, 2 points if in gender, 1 points if in gender/diagnosis). The total score can be up to 36 points: 24 from our empirical data, and 12 from literature data used for CFG. We weigh our new empirical data more than the literature data, as it is functionally related to mood in 3 independent cohorts (discovery, validation, testing). The goal is to highlight, based on the totality of our data and of the evidence in the field to date, biomarkers that have all around evidence: track anxiety, have convergent evidence for involvement in anxiety disorders, and predict anxiety state and future clinical events (Table 2). Such biomarkers merit priority evaluation in future clinical trials. The top biomarkers (n=19) are depicted in Table 2.

### Targeted Therapeutics

### Pharmacogenomics.

A number of individual top biomarkers are known to be modulated by medications in current clinical use and by nutraceuticals (Table 6, FIG. 4). This is of potential utility in patient stratification and pharmacogenomics approaches. Omega-3 fatty acids may be a widely deployable preventive treatment, with minimal side-effects, including in women who are or may become pregnant.

### New Drug Discovery/Repurposing.

Bioinformatic analyses using the gene expression signature of the panel of top biomarkers for anxiety (Table 4) identified new potential therapeutics for anxiety, such as estrogen.

TABLE 1
Aggregate Demographics, BP—bipolar, MDD—depression, MOOD—mood nos, SZ—schizophrenia,
SZA—schizoaffective, PSYCH—psychosis nos, PTSD—post-traumatic stress disorder.

| | | | | | |
|---|---|---|---|---|---|
| Number | | | | | |
| of | | | | Age Mean | |
| Subjects | Gender | Diagnosis | Ethnicity | (SD) | T-test for age |

Discovery

| | | | | | |
|---|---|---|---|---|---|
| Discovery Cohort | 58 | Male = | BP = 18(52) | EA = 41 | All = 49.6 |
| (Longitudinal | (149 visits) | 41 | MDD = 10(23) | AA = 14 | (8.63) |
| Within-Subject | | Female = | SZA = 14(34) | Mixed = | Low Anxiety = |
| Changes | | 17 | SZ = 7(17) | 1 | 49.7 (8.77) |
| Anxiety Levels | | | PTSD = 7(17) | Asian | High Anxiety = |
| (SAS-4) | | | Mood = 2(6) | American = | 49.4 (8.51) |
| | | | | 1 | |
| | | | | Hispanic = | |
| | | | | 1 | |

Validation

| | | | | | | |
|---|---|---|---|---|---|---|
| Independent | 40 | Male = | BP = 15(18) | EA = 29 | 47.9 | Discovery vs. |
| Validation Cohort | (60 visits) | 32 | MDD = 6(9) | AA = 11 | (8.4) | Validation = |
| for Gene | | Female = | SZA = 6(10) | | | 0.20636 |
| Expression | | 8 | SZ = 3(7) | | | |
| (Clinically Severe | | | PTSD = 8(14) | | | |
| Anxiety) | | | MOOD = 2(2) | | | |
| SAS-4 (≥60) | | | | | | |
| STAI (≥55) | | | | | | |

Testing

| | | | | | | |
|---|---|---|---|---|---|---|
| Independent | 197 | Male = | BP = 60 (171) | EA = 132 | All = 50.07 | T-test for age |
| Testing Cohort For | (495 visits) | 161 | MDD = 34 (78) | AA = 62 | (9.7) | between High |
| Predicting State | | Female = | SZA = 42 (100) | Hispanic = | High Anxiety = | Anxiety (SAS-4) |
| (Anxiety Levels at | | 36 | SZ = 40 (101) | 2 | 48.6 (n = 19) | vs. Others |
| Time of | | | PTSD = 13 (26) | Asian = 1 | Others = 50.1 | 0.542 |
| Assessment) | | | MOOD = 3 (8) | | (n = 476) | |
| Anxiety SAS-4 | | | PSYCH = 5 (11) | | | |
| (≥60) | | | | | | |
| Independent | 195 | Male = | BP = 60(170) | EA = 131 | All = 50.04 | T-test for age |
| Testing Cohort For | (486 visits) | 159 | MDD = 34(78) | AA = 61 | (9.7) | between High |
| Predicting State | | Female = | SZA = 41(95) | Hispanic = | High Anxiety | Anxiety (STAI) |
| (Anxiety Levels at | | 36 | SZ = 39(98) | 2 | (STAI) = 50.9 | vs. Others |
| Time of | | | PTSD = 13(26) | Asian = 1 | (n = 42) | 0.9673 |
| Assessment) | | | MOOD = 3(8) | | Others = 50 | |
| Anxiety STAI (≥55) | | | PSYCH = 5(11) | | (n = 444) | |
| Independent Testing | 101 | Male = | BP = 34 (94) | EA = 73 | 44.5 | T-test for age |
| Cohort | (236 Visits) | 86 | MDD = 19(40) | AA = 26 | (8.11) | Hosp with |
| For Predicting Trait | | Female = | SZA = 17 (38) | Hispanic = | Anxiety Hosp = | Anxiety vs. |
| (Hospitalization | | 15 | SZ = 22 (49) | 2 | 43 (n = 18) | others |
| with Anxiety in the | | | PTSD = 9 (15) | | Others = 44.5 | First year |
| First Year | | | | | (n = 218) | p = 0.59098 |
| Following | | | | | | |
| Assessment) | | | | | | |
| Independent Testing | 130 | Male = | BP = 39 (107) | EA = 90 | 47.4 | T-test for age |
| Cohort | (318 Visits) | 118 | MDD = 28 (59) | AA = 44 | (5.64) | Hosp with no |
| For Predicting Trait | | Female = | SZA = 28 (64) | Hispanic = | Anxiety Future | Anxiety vs. vs. |
| (All Future | | 18 | SZ = 31 (70) | 2 | Hosp = 47.2 | Others |
| Hospitalizations | | | PTSD = 10 (18) | | (n = 70) | All Future Years |
| with Anxiety) | | | | | Others = 47.5 | 0.842276 |
| | | | | | (n = 248) | |

TABLE 2
Top Anxiety Biomarkers: Convergent Functional Evidence (CFE). After Step 4 Testing
in independent cohorts for state and trait predictive ability. For Step 4 Predictions,
C:—cross-sectional (using levels from one visit), L:—longitudinal (using levels
and slopes from multiple visits). In ALL, by Gender, and personalized by Gender
and Diagnosis (Gender/Dx). M—Males, F—Females. MDD—depression, BP—bipolar,
SZ—schizophrenia, SZA—schizoaffective, PSYCHOSIS—schizophrenia and schizoaffective
combined, PTSD—post-traumatic stress disorder.

| | | | Step 2 | | Step 4 | Step 4 |
|---|---|---|---|---|---|---|
| | | | External | | Significant | Significant |
| | | Step 1 | Convergent | | Predictions | Predictions |
| | | Discovery | Functional | | of High | of High |
| | | (Direc- | Genomics | | Anxiety | STAI |
| | | tion of | (CFG) | Step 3 | State | State |
| | | Change | Evidence | Vali- | ROC AUC/ | ROC AUC/ |
| | | in High | For | dation | p-value | p-value |
| | | Anxiety) | Involvement | ANOVA | 3 pts | 3 pts |
| | | Method/ | in | p- | ALL 2 pts | ALL 2 pts |
| Symbol/ | | Score/ | Anxiety | value/ | Gender | Gender |
| Gene | Probe- | % | Score | Score | 1 pts | 1 pts |
| Name | sets | 6 pts | 12 pts | 6 pts | Gender/ Dx | Gender/ Dx |
| GAD1 | 205278_{—} | (I) | 11 | 6.79E−01/0 | | ALL |
| Glutamate | at | DE/4 | | Not | | C: |
| Decar- | | 62.3% | | Stepwise | | (42/486) |
| boxylase | | | | | | 0.58/3.94E−02 |
| 1 | | | | | | Gender |
| | | | | | | Females |
| | | | | | | C: |
| | | | | | | (17/88) |
| | | | | | | 0.65/2.75E−02 |
| | | | | | | Gender-dx |
| | | | | | | F- |
| | | | | | | PSYCHOSIS |
| | | | | | | C: |
| | | | | | | (5/28) |
| | | | | | | 0.77/2.94E−02 |
| | | | | | | F-SZ |
| | | | | | | C: |
| | | | | | | (4/8) |
| | | | | | | 0.94/2.17E−02 |
| NTRK3 | 215311_{—} | (I) | 4 | 3.26E−01/2 | Gender | ALL |
| Neurotro- | at | DE/4 | | Stepwise | Males | C: |
| phic | | 53.2% | | | L: | (42/486) |
| Receptor | | | | | (4/246) | 0.59/3.04E−02 |
| Tyrosine | | | | | 0.77/2.98E−02 | L: |
| Kinase | | | | | Gender-dx | (21/291) |
| 3 | | | | | M- | 0.61/4.83E−02 |
| | | | | | PSYCHOSIS | Gender |
| | | | | | L: | Females |
| | | | | | (2/102) | C: |
| | | | | | 0.9/2.83E−02 | (17/88) |
| | | | | | M-SZA | 0.68/1.20E−02 |
| | | | | | L: | L: |
| | | | | | (2/46) | (10/52) |
| | | | | | 0.85/4.75E−02 | 0.7/2.85E−02 |
| | | | | | | Gender-dx |
| | | | | | | F-BP |
| | | | | | | C: (4/31) |
| | | | | | | 0.89/6.66E−03 |
| | | | | | | M-MDD |
| | | | | | | L: (4/31) |
| | | | | | | 0.82/1.96E−02 |
| ADRA2A | 209869_{—} | (I) | 4 | 9.07E−01/0 | Gender-dx | Gender |
| Adreno- | at | AP/4 | | Not | M-MDD | Females |
| ceptor | | 50% | | Stepwise | C: | C: |
| Alpha | | | | | (2/57) | (17/88) |
| 2A | | | | | 0.88/3.43E−02 | 0.63/4.91E−02 |
| | | | | | | F-SZ |
| | | | | | | C: |
| | | | | | | (4/8) |
| | | | | | | 0.88/4.07E−02 |
| | | | | | | L: (3/5) |
| | | | | | | 1/4.16E−02 |
| FZD10 | 219764_{—} | (I) | 2 | 3.57E−01/2 | | ALL |
| Frizzled | at | DE/4 | | Stepwise | | C: |
| Class | | 53.2% | | | | (42/486) |
| Receptor | | | | | | 0.6/1.39E−02 |
| 10 | | | | | | L: |
| | | | | | | (21/291) |
| | | | | | | 0.64/1.43E−02 |
| | | | | | | Gender |
| | | | | | | Females |
| | | | | | | C: |
| | | | | | | (17/88) |
| | | | | | | 0.69/7.42E−03 |
| | | | | | | L: |
| | | | | | | (10/52) |
| | | | | | | 0.68/3.88E−02 |
| | | | | | | F-BP |
| | | | | | | C: (4/31) |
| | | | | | | 0.85/1.26E−02 |
| | | | | | | F-SZA |
| | | | | | | C: (1/20) |
| | | | | | | 1/4.97E−02 |
| | | | | | | F-SZ |
| | | | | | | L: (3/5) |
| | | | | | | 1/4.16E−02 |
| GRK4G | 210600_{—} | (I) | 0 | 3.40E−01/2 | Gender-dx | Gender |
| Protein- | s_at | DE/6 | | Stepwise | M-MDD | Females |
| Coupled | | 81.8% | | | C: | C: |
| Receptor | | | | | (2/57) | (17/88) |
| Kinase | | | | | 0.87/3.77E−02 | 0.64/3.26E−02 |
| | | | | | | Gender-dx |
| | | | | | | F-BP |
| | | | | | | C: |
| | | | | | | (4/31) |
| | | | | | | 0.86/1.08E−02 |
| | | | | | | M-MDD |
| | | | | | | L: |
| | | | | | | (4/31) |
| | | | | | | 0.85/1.26E−02 |
| ATP1B2 | 204311_{—} | (I) | 4 | 1.65E−01/2 | Gender-dx | Gender |
| ATPase | at | DE/2 | | Stepwise | M-MDD | Females |
| Na+/K+ | | 39% | | | C: | C: |
| Trans- | | | | | (2/57) | (17/88) |
| porting | | | | | 0.9/2.81E−02 | 0.64/3.75E−02 |
| Subunit | | | | | | Gender-dx |
| | | | | | | F-BP |
| | | | | | | C: |
| | | | | | | (4/31) |
| | | | | | | 0.84/1.46E−02 |
| | | | | | | F- |
| | | | | | | PSYCHOSIS |
| | | | | | | C: |
| | | | | | | (5/28) |
| | | | | | | 0.77/3.37E−02 |
| CLIC6 | 242913_{—} | (I) | 2 | 5.06E−01/0 | | Gender |
| Chloride | at | AP/6 | | Not | | Females |
| Intracel- | | 84.2% | | Stepwise | | C: |
| lular | | | | | | (17/88) |
| Channel 6 | | | | | | 0.67/1.68E−02 |
| | | | | | | Gender-dx |
| | | | | | | F-BP |
| | | | | | | C: |
| | | | | | | (4/31) |
| | | | | | | 0.8/2.97E−02 |
| | | | | | | F- |
| | | | | | | PSYCHOSIS |
| | | | | | | C: (5/28) |
| | | | | | | 0.85/8.20E−03 |
| | | | | | | F-SZ |
| | | | | | | C: (4/8) |
| | | | | | | 0.97/1.47E−02 |
| | | | | | | F-SZA |
| | | | | | | C: (1/20) |
| | | | | | | 1/4.97E−02 |
| EFNA5 | 1559360_{—} | (I) | 2 | 2.02E−04/4 | | Gender |
| Ephrin | at | DE/6 | | Nominal | | Females |
| A5 | | 80.5% | | | | C: |
| | | (I) | | | | (17/88) |
| | | AP/4 | | | | 0.63/4.85E−02 |
| | | 51.3% | | | | Gender-dx |
| | | | | | | M-SZ |
| | | | | | | C: |
| | | | | | | (6/90) |
| | | | | | | 0.71/4.03E−02 |
| GPX7 | 213170_{—} | (D) | 2 | 4.64E−01/2 | ALL | Gender-dx |
| Glutathi- | at | DE/4 | | Stepwise | C: | F-BP |
| one | | 64.3% | | | (19/495) | C: |
| Peroxi- | | | | | 0.63/2.63E−02 | (4/31) |
| dase 7 | | | | | | 0.86/1.08E−02 |
| | | | | | | F-SZA |
| | | | | | | C: |
| | | | | | | (1/20) |
| | | | | | | 1/4.97E−02 |
| | | | | | | M-MDD |
| | | | | | | L: |
| | | | | | | (4/31) |
| | | | | | | 0.86/1.08E−02 |
| NTRK3 | 215025_{—} | (I) | 4 | 6.58E−01/2 | Gender-dx | ALL |
| Neuro- | at | DE/2 | | Stepwise | M-BP | C: |
| trophic | | 35.1% | | | L: | (42/486) |
| Receptor | | | | | (2/93) | 0.61/9.05E−03 |
| Tyrosine | | | | | 0.9/2.83E−02 | L: |
| Kinase | | | | | | (21/291) |
| 3 | | | | | | 0.64/1.43E−02 |
| | | | | | | Gender |
| | | | | | | Females |
| | | | | | | C: |
| | | | | | | (17/88) |
| | | | | | | 0.72/2.51E−03 |
| | | | | | | Males |
| | | | | | | L: |
| | | | | | | (11/239) |
| | | | | | | 0.67/2.55E−02 |
| | | | | | | Gender-dx |
| | | | | | | F-BP |
| | | | | | | C: |
| | | | | | | (4/31) |
| | | | | | | 0.86/1.08E−02 |
| | | | | | | F- |
| | | | | | | PSYCHOSIS |
| | | | | | | C: (5/28) |
| | | | | | | 0.77/2.93E−02 |
| | | | | | | F-SZ |
| | | | | | | C: (4/8) |
| | | | | | | 0.94/1.92E−02 |
| | | | | | | M-BP |
| | | | | | | L: |
| | | | | | | (2/92) |
| | | | | | | 0.93/1.84E−02 |
| SLC6A2 | 217214_{—} | (I) | 4 | 3.40E−01/2 | Gender-dx | ALL |
| Solute | s_at | DE/2 | | Stepwise | F- | C: |
| Carrier | | 40.3% | | | PSYCHOSIS | (42/486) |
| Family | | | | | L: | 0.63/2.37E−03 |
| 6 | | | | | (4/17) | L: |
| Member | | | | | 0.81/3.50E−02 | (21/291) |
| 2 | | | | | F-SZA | 0.62/3.78E−02 |
| | | | | | L: | Gender |
| | | | | | (4/12) | Females |
| | | | | | 0.81/4.47E−02 | C: |
| | | | | | | (17/88) |
| | | | | | | 0.76/4.78E−04 |
| | | | | | | Gender-dx |
| | | | | | | F-MDD |
| | | | | | | C: |
| | | | | | | (7/21) |
| | | | | | | 0.77/2.62E−02 |
| | | | | | | F- |
| | | | | | | PSYCHOSIS |
| | | | | | | C: |
| | | | | | | (5/28) |
| | | | | | | 0.8/1.92E−02 |
| | | | | | | F-SZ |
| | | | | | | C: |
| | | | | | | (4/8) |
| | | | | | | 0.94/2.17E−02 |
| | | | | | | M-SZ |
| | | | | | | C: |
| | | | | | | (6/90) |
| | | | | | | 0.71/4.63E−02 |
| SLC6A4 | 242009_{—} | (I) | 10 | 8.51E−01/0 | | |
| Solute | at | DE/2 | | Not | | |
| Carrier | | 35.1% | | Stepwise | | |
| Family | | | | | | |
| 6 | | | | | | |
| Member | | | | | | |
| 4 | | | | | | |
| TMEM138 | 223113_{—} | (D) | 4 | 8.63E−01/0 | | Gender |
| Trans- | at | DE/4 | | Not | | Males |
| membrane | | 63% | | Stepwise | | L: |
| Protein | | | | | | (11/239) |
| 138 | | | | | | 0.66/4.12E−02 |
| | | | | | | Gender-dx |
| | | | | | | F-BP |
| | | | | | | C: |
| | | | | | | (4/31) |
| | | | | | | 0.86/1.08E−02 |
| ANKRD28 | 229307_{—} | (I) | 0 | 6.20E−01/2 | | ALL |
| Ankyrin | at | DE/6 | | Stepwise | | C: |
| Repeat | | 80.5% | | | | (42/486) |
| Domain | | | | | | 0.62/5.30E−03 |
| 28 | | | | | | Gender |
| | | | | | | Males |
| | | | | | | C: |
| | | | | | | (25/398) |
| | | | | | | 0.72/1.31E−04 |
| | | | | | | Gender-dx |
| | | | | | | M-MDD |
| | | | | | | C: |
| | | | | | | (7/57) |
| | | | | | | 0.89/3.97E−04 |
| | | | | | | Gender-dx |
| | | | | | | M- |
| | | | | | | PSYCHOSIS |
| | | | | | | C: |
| | | | | | | (12/165) |
| | | | | | | 0.72/5.72E−03 |
| | | | | | | Gender-dx |
| | | | | | | M-SZ |
| | | | | | | C: |
| | | | | | | (6/90) |
| | | | | | | 0.84/3.13E−03 |
| CCKBR | 210381_{—} | (I) | 4 | 4.37E−01/2 | | Gender-dx |
| Cholecy | s_at | DE/2 | | Stepwise | | F-BP |
| stokinin | | 48% | | | | C: |
| B | | | | | | (4/31) |
| Receptor | | | | | | 0.8/2.97E−02 |
| | | | | | | Gender-dx |
| | | | | | | M-BP |
| | | | | | | C: |
| | | | | | | (5/139) |
| | | | | | | 0.74/3.70E−02 |
| | | | | | | Gender-dx |
| | | | | | | F-BP |
| | | | | | | L: |
| | | | | | | (3/18) |
| | | | | | | 0.82/4.29E−02 |
| DYNLL2 | 229106_{—} | (D) | 4 | 1.96E−01/2 | Gender | Gender-dx |
| Dynein | at | DE/2 | | Stepwise | Males | F-SZA |
| Light | | 39.3% | | | L: | C: |
| Chain | | | | | (4/246) | (1/20) |
| LC8- | | | | | 0.78/2.79E−02 | 1/4.97E−02 |
| Type 2 | | | | | Gender-dx | |
| | | | | | M-BP | |
| | | | | | L: (2/93) | |
| | | | | | 0.85/4.50E−02 | |
| Hs.550187 | 240253_{—} | (I) | 0 | 2.74E−01/2 | | |
| | at | DE/6 | | Stepwise | | |
| | | 88.3% | | | | |
| NRG1 | 208232_{—} | (I) | 4 | 6.66E−01/2 | Gender-dx | Gender-dx |
| Neuregu- | x_at | DE/4 | | Stepwise | M-MDD | F-BP |
| lin 1 | | 65.8% | | | C: | C: |
| | | | | | (2/57) | (4/31) |
| | | | | | 0.93/2.08E−02 | 0.81/2.59E−02 |
| | | | | | M-BP | Gender-dx |
| | | | | | L: | M- |
| | | | | | (2/93) | PSYCHOSIS |
| | | | | | 0.85/4.50E−02 | C: |
| | | | | | | (12/165) |
| | | | | | | 0.73/3.69E−03 |
| | | | | | | M-SZ |
| | | | | | | C: |
| | | | | | | (6/90) |
| | | | | | | 0.7/4.79E−02 |
| | | | | | | M-SZA |
| | | | | | | C: |
| | | | | | | (6/75) |
| | | | | | | 0.76/1.66E−02 |
| TFRC | 207332_{—} | (D) | 2.00 | 7.11E−01/2 | Gender | Gender-dx |
| Trans- | s_at | DE/4 | | Stepwise | Females | F-BP |
| ferrin | | 56% | | | L: | C: |
| Receptor | | | | | (4/52) | (4/31) |
| | | | | | 0.82/1.66E−02 | 0.88/7.85E−03 |
| | | | | | Gender-dx | |
| | | | | | F- | |
| | | | | | PSYCHOSIS | |
| | | | | | C: | |
| | | | | | (4/28) | |
| | | | | | 0.77/4.39E−02 | |
| | | | | | L: | |
| | | | | | (4/17) | |
| | | | | | 0.85/2.08E−02 | |
| | | | | | F-SZA | |
| | | | | | L: | |
| | | | | | (4/12) | |
| | | | | | 0.84/3.09E−02 | |

| | Step 4 | Step 4 | | | CFE |
|---|---|---|---|---|---|
| | Significant | Significant | | | Polyevi- |
| | Predictions | Predictions | | | dence |
| | of First | of Future | | Drugs | Score |
| | Year Hosp. | Hosp for | | that | for |
| | for Anxiety | Anxiety | | Modulate | Involve- |
| | ROC AUC/ | OR/p- | | the | ment |
| | p-value | value | Other | Biomarker | in |
| | 3 pts | 3 pts | Psychiatric | in | Anxiety |
| | ALL 2 pts | ALL 2 pts | and | Opposite | (Based |
| Symbol/ | Gender | Gender | Related | Direction | on |
| Gene | 1 pts | 1 pts | Disorders | to High | Steps |
| Name | Gender /Dx | Gender /Dx | Evidence | Anxiety | 1-4) |
| GAD1 | Gender-dx | ALL | Alcohol | Carba- | 22 |
| Glutamate | M- | C: | Withdrawal | mazepine | |
| Decar- | PSYCHOSIS | (70/38) | Autism | Lithium | |
| boxylase | C: | 1.3/5.52E−03 | BP | Omega-3 | |
| 1 | (7/74) | Gender | Creative | fatty | |
| | 0.69/4.73E−02 | Males | reliable | acids | |
| | M-SZA | C: | Depression | Valproate | |
| | C: | (59/273) | Intellectual | | |
| | (3/32) | 1.34/2.80E−03 | Disability | | |
| | 0.79/4.96E−02 | Gender-dx | MDD | | |
| | | M-BP | Mood | | |
| | | C: (13/96) | Disorders | | |
| | | 1.69/2.12E−02 | NOS | | |
| | | M- | Phencycli- | | |
| | | PSYCHOSIS | dine | | |
| | | C: | PTSD | | |
| | | (37/121) | Suicide | | |
| | | 1.33/9.36E−03 | SZ | | |
| | | M-SZA | | | |
| | | C: (23/58) | | | |
| | | 1.58/9.14E−03 | | | |
| NTRK3 | Gender-dx | ALL | Aging | Clozapine | 19 |
| Neurotro- | F- | C: | Alcohol | | |
| phic | PSYCHOSIS | (70/318) | Alzheimer's | | |
| Receptor | C: | 1.23/9.38E−03 | Disease | | |
| Tyrosine | (2/13) | Gender | Bipolar II | | |
| Kinase | 0.91/3.78E−02 | Females | BP | | |
| 3 | Gender-dx | C: | Longevity | | |
| | F-SZA | (11/45) | MDD Mood | | |
| | C: | 1.84/1.60E−02 | Disorders | | |
| | (2/6) | | NOS Pain | | |
| | 1/3.20E−02 | | PTSD | | |
| | | | Risky | | |
| | | | Behavior | | |
| | | | SAD | | |
| | | | Social | | |
| | | | Isolation | | |
| | | | Stress | | |
| | | | Subsyndromal | | |
| | | | symptomatic | | |
| | | | depression | | |
| | | | Suicide SZ | | |
| ADRA2A | ALL | ALL | Alcohol | Carba- | 17 |
| Adreno- | C: | C: | Bipolar | mazepine | |
| ceptor | (18/236) | (70/318) | COVID-19 | Clozapine | |
| Alpha | 0.65/1.80E−02 | 1.41/4.65E−04 | Depression | Norflu- | |
| 2A | Gender | Gender | MDD | oxetine | |
| | Males | Males | Pain | Valproate | |
| | C: | C: | PTSD | | |
| | (15/199) | (59/273) | Suicide | | |
| | 0.67/1.44E−02 | 1.44/5.74E−04 | SZ | | |
| | Gender-dx | Gender-dx | | | |
| | M- | M- | | | |
| | PSYCHOSIS | PSYCHOSIS | | | |
| | C: | C: | | | |
| | (7/74) | (37/121) | | | |
| | 0.81/3.22E−03 | 1.52/3.60E−03 | | | |
| | M-PTSD | M-PTSD | | | |
| | C: | C: | | | |
| | (3/10) | (5/12) | | | |
| | 0.86/4.37E−02 | 2.59/2.08E−02 | | | |
| | M-SZ | M-SZ | | | |
| | C: | C: | | | |
| | (4/42) | (14/63) | | | |
| | 0.91/3.47E−03 | 2.14/1.61E−04 | | | |
| FZD10 | ALL | ALL | Alcohol | Aripipra- | 17 |
| Frizzled | C: | C: | ALZ | zole | |
| Class | (18/236) | (70/318) | BP | Fluox- | |
| Receptor | 0.67/7.66E−03 | 1.34/3.63E−03 | Circadian | etine | |
| 10 | Gender | Gender | abnormal- | Gamma | |
| | Males | Males | ities | Frequency | |
| | C: | C: | MDD | | |
| | (15/199) | (59/273) | PTSD | | |
| | 0.68/1.22E−02 | 1.34/6.61E−03 | | | |
| | L: | Gender-dx | | | |
| | (4/113) | M- | | | |
| | 0.75/4.52E−02 | PSYCHOSIS | | | |
| | Gender-dx | C: | | | |
| | M- | (37/121) | | | |
| | PSYCHOSIS | 1.55/2.70E−03 | | | |
| | C: (7/74) | M-SZ | | | |
| | 0.75/1.64E−02 | C: | | | |
| | L: (1/39) | (14/63) | | | |
| | 1/4.57E−02 | 1.92/5.16E−03 | | | |
| | M-SZ | | | | |
| | C: (4/42) | | | | |
| | 0.87/8.21E−03 | | | | |
| | L: (1/22) | | | | |
| | 1/4.90E−02 | | | | |
| GRK4G | ALL | ALL | Autism | Clozapine | 17 |
| Protein- | C: | C: | Bipolar | Gamma | |
| Coupled | (18/236) | (70/318) | Cannabis | Frequency | |
| Receptor | 0.62/4.27E−02 | 1.32/4.46E−03 | Depression | | |
| Kinase | Gender | Gender | PTSD | | |
| | Males | Males | Stress | | |
| | C: | C: | | | |
| | (15/199) | (59/273) | | | |
| | 0.64/4.06E−02 | 1.32/8.99E−03 | | | |
| | | Gender-dx | | | |
| | | F-MDD | | | |
| | | C: (2/15) | | | |
| | | 1.87/4.51E−02 | | | |
| | | Gender-dx | | | |
| | | M- | | | |
| | | PSYCHOSIS | | | |
| | | C: (37/121) | | | |
| | | 1.34/2.11E−02 | | | |
| ATP1B2 | Gender | ALL | Alcohol | Acetyl | 16 |
| ATPase | Males | C: | ALZ | digitoxin | |
| Na+/K+ | C: | (70/318) | MDD | Clozapine | |
| Trans- | (15/199) | 1.59/1.91E−05 | PTSD | Deslano- | |
| porting | 0.63/4.18E−02 | Gender | Stress | side | |
| Subunit | Gender-dx | Females | Substance | Digitoxin | |
| | M- | C: | Abuse | Digoxin | |
| | PSYCHOSIS | (11/45) | Suicide | Haloperi- | |
| | C: | 1.68/2.97E−02 | SZ | dol | |
| | (7/74) | Males | | Lithium | |
| | 0.79/5.55E−03 | C: | | Valproate | |
| | M-PTSD | (59/273) | | | |
| | C: | 1.56/1.33E−04 | | | |
| | (3/10) | Gender-dx | | | |
| | 0.86/4.37E−02 | F-MDD | | | |
| | M-SZ | C: (2/15) | | | |
| | C: | 3.16/3.10E−02 | | | |
| | (4/42) | M-BP | | | |
| | 0.86/9.22E−03 | C: (13/96) | | | |
| | | 1.61/1.79E−02 | | | |
| | | M- | | | |
| | | PSYCHOSIS | | | |
| | | C: (37/121) | | | |
| | | 1.98/4.84E−05 | | | |
| | | M-SZ | | | |
| | | C: (14/63) | | | |
| | | 2.39/7.75E−04 | | | |
| | | M-SZA | | | |
| | | C: (23/58) | | | |
| | | 1.68/1.24E−02 | | | |
| CLIC6 | ALL | ALL | Aging | Clozapine | 16 |
| Chloride | C: | C: | Alcohol | Omega-3 | |
| Intracel- | (18/236) | (70/318) | Cocaine | fatty | |
| lular | 0.62/4.11E−02 | 1.32/6.89E−03 | MDD | acids | |
| Channel 6 | Gender-dx | Gender | Phencycli- | Gamma | |
| | M- | Females | dine | Frequency | |
| | PSYCHOSIS | C: | Restraint | | |
| | C: | (11/45) | Stress | | |
| | (7/74) | 1.59/4.32E−02 | Suicide | | |
| | 0.71/3.17E−02 | Males | | | |
| | Gender-dx | C: | | | |
| | M-SZ | (59/273) | | | |
| | C: | 1.27/2.51E−02 | | | |
| | (4/42) | Gender-dx | | | |
| | 0.8/2.44E−02 | M-SZ | | | |
| | | C: | | | |
| | | (14/63) | | | |
| | | 1.68/1.04E−02 | | | |
| EFNA5 | ALL | Gender-dx | Alcohol | Omega-3 | 16 |
| Ephrin | L: | M-BP | ALZ | fatty | |
| A5 | (5/133) | C: | BP | acids | |
| | 0.74/3.34E−02 | (13/96) | Cannabis | Gamma | |
| | Gender | 1.41/2.92E−02 | Chronic | Frequency | |
| | Males | | Fatigue | | |
| | L: | | Syndrome | | |
| | (4/113) | | Depression | | |
| | 0.75/4.67E−02 | | Insomnia | | |
| | Gender-dx | | Intellect | | |
| | F-SZA | | Longevity | | |
| | C: | | MDD | | |
| | (2/6) | | Morphine | | |
| | 1/3.20E−02 | | Pain | | |
| | M-SZA | | Stress | | |
| | C: (3/32) | | Suicide | | |
| | 0.79/4.96E−02 | | SZ | | |
| GPX7 | Gender-dx | ALL | Aging | Mianserin | 16 |
| Glutathione | M- | C: | MDD | S- | |
| Peroxidase | PSYCHOSIS | (70/318) | Neuropathic | adenosyl | |
| 7 | C: | 1.42/6.50E−03 | pain | methio- | |
| | (7/74) | Gender | SZA | nine | |
| | 0.71/3.17E−02 | Males | | (SAM) | |
| | M-SZ | C: | | | |
| | C: | (59/273) | | | |
| | (4/42) | 1.47/7.12E−03 | | | |
| | 0.82/1.99E−02 | Gender-dx | | | |
| | | M- | | | |
| | | PSYCHOSIS | | | |
| | | C: | | | |
| | | (37/121) | | | |
| | | 1.5/2.40E−02 | | | |
| | | M-SZ | | | |
| | | C: | | | |
| | | (14/63) | | | |
| | | 1.9/4.95E−02 | | | |
| NTRK3 | Gender-dx | ALL | Aging | Clozapine | 16 |
| Neuro- | M- | C: | Alcohol | | |
| trophic | PSYCHOSIS | (70/318) | Alzheimer's | | |
| Receptor | C: | 1.36/5.70E−03 | Disease | | |
| Tyrosine | (7/74) | Gender | Bipolar I | | |
| Kinase | 0.7/4.55E−02 | Males | BP | | |
| 3 | Gender-dx | C: | Depression | | |
| | M-SZ | (59/273) | Longevity | | |
| | C: | 1.4/4.76E−03 | MDD | | |
| | (4/42) | Gender-dx | Mood | | |
| | 0.82/1.99E−02 | M-BP | Disorders | | |
| | L: | C: | NOS | | |
| | (1/22) | (13/96) | Pain | | |
| | 1/4.90E−02 | 1.95/1.24E−02 | PTSD | | |
| | | M- | Risky | | |
| | | PSYCHOSIS | Behavi | | |
| | | C: | SAD | | |
| | | (37/121) | Social | | |
| | | 1.4/1.87E−02 | Isolation | | |
| | | M-SZ | Stress | | |
| | | C: | Subsyndromal | | |
| | | (14/63) | symptomatic | | |
| | | 1.93/3.78E−03 | depression | | |
| | | | Suicide | | |
| | | | SZ | | |
| SLC6A2 | Gender-dx | ALL | Alcohol | Fluox- | 16 |
| Solute | M- | C: | Depression | etine | |
| Carrier | PSYCHOSIS | (70/318) | MDD | Norflu- | |
| Family | C: | 1.33/4.74E−03 | Postpartum | oxetine | |
| 6 | (7/74) | Gender | Depression | | |
| Member | 0.72/2.92E−02 | Males | PTSD | | |
| 2 | M-SZA | C: | Suicide | | |
| | C: | (59/273) | | | |
| | (3/32) | 1.34/6.73E−03 | | | |
| | 0.87/1.78E−02 | Gender-dx | | | |
| | M- | M-BP | | | |
| | PSYCHOSIS | C: | | | |
| | L: | (13/96) | | | |
| | (1/39) | 1.61/4.50E−02 | | | |
| | 1/4.57E−02 | M- | | | |
| | M-SZ | PSYCHOSIS | | | |
| | L: | C: | | | |
| | (1/22) | (37/121) | | | |
| | 1/4.90E−02 | 1.31/2.54E−02 | | | |
| SLC6A4 | Gender-dx | ALL | Aging | Agomela- | 16 |
| Solute | F- | C: | Alcohol | tine | |
| Carrier | PSYCHOSIS | (70/318) | Anxiety | Benzodi- | |
| Family | C: | 1.21/1.15E−02 | BPD | azepines | |
| 6 | (2/13) | Gender | Depression | Citalo- | |
| Member | 0.91/3.76E−02 | Males | Early | pram | |
| 4 | F-SZA | C: | Life | Clozapine | |
| | C: | (59/273) | Stress | Omega-3 | |
| | (2/6) | 1.19/2.77E−02 | MDD | fatty | |
| | 1/3.20E−02 | Gender-dx | MSK | acids | |
| | M- | M- | Neuro- | Oxycodone | |
| | PTSD | PTSD | pathic | Sertra- | |
| | C: | C: | pain | line | |
| | (3/10) | (5/12) | Pain | Vortiox- | |
| | 0.86/4.37E−02 | 2.54/3.24E−02 | Pain | etine | |
| | M-SZ | | Postpartum | | |
| | C: | | Depression | | |
| | (4/42) | | PTSD | | |
| | 0.76/4.74E−02 | | Stress | | |
| | | | Suicide | | |
| TMEM138 | ALL | ALL | Alcohol | Antide- | 16 |
| Trans- | L: | C: | Brain | pressants | |
| membrane | (5/129) | (70/318) | arousal | | |
| Protein | 0.72/4.61E−02 | 1.3/1.46E−02 | Depression | | |
| 138 | Gender-dx | Gender | | | |
| | M- | Males | | | |
| | PTSD | C: | | | |
| | C: | (59/273) | | | |
| | (3/10) | 1.34/1.20E−02 | | | |
| | 0.86/4.37E−02 | Gender-dx | | | |
| | | M- | | | |
| | | PSYCHOSIS | | | |
| | | C: | | | |
| | | (37/121) | | | |
| | | 1.5/6.57E−03 | | | |
| | | M-PTSD | | | |
| | | C: | | | |
| | | (5/12) | | | |
| | | 3.71/4.13E−02 | | | |
| | | M-SZA | | | |
| | | C: | | | |
| | | (23/58) | | | |
| | | 1.51/2.83E−02 | | | |
| ANKRD28 | Gender-dx | ALL | Alcohol | N/A | 15 |
| Ankyrin | F- | C: | ASD | | |
| Repeat | PSYCHOSIS | (70/318) | BP | | |
| Domain | C: | 1.22/2.96E−02 | Childhood | | |
| 28 | (2/13) | Gender-dx | Trauma | | |
| | 0.91/3.76E−02 | F-MDD | Depression | | |
| | Gender-dx | C: | MDD | | |
| | F-SZA | (2/15) | Mood | | |
| | C: | 3.09/3.59E−02 | instability | | |
| | (2/6) | Gender | PTSD | | |
| | 1/3.20E−02 | Males | Stress | | |
| | | C: | Suicide | | |
| | | (59/273) | | | |
| | | 1.21/4.06E−02 | | | |
| | | Gender-dx | | | |
| | | M- | | | |
| | | PSYCHOSIS | | | |
| | | C: | | | |
| | | (37/121) | | | |
| | | 1.42/7.30E−03 | | | |
| | | Gender-dx | | | |
| | | M-SZA | | | |
| | | C: | | | |
| | | (23/58) | | | |
| | | 1.61/1.24E−03 | | | |
| CCKBR | ALL | ALL | Alcohol | Clozapine | 15 |
| Cholecysto- | C: | C: | BP | | |
| kinin | (18/236) | (70/318) | Chronic | | |
| B | 0.63/3.01E−02 | 1.33/4.28E−03 | Stress | | |
| Receptor | Gender-dx | Gender | MDD | | |
| | F- | Males | Phencycli- | | |
| | PSYCHOSIS | C: | dine | | |
| | C: | (59/273) | Suicide | | |
| | (2/13) | 1.32/9.50E−03 | SZ | | |
| | 0.91/3.78E−02 | Gender-dx | | | |
| | Gender-dx | M- | | | |
| | F-SZA | PSYCHOSIS | | | |
| | C: | C: | | | |
| | (2/6) | (37/121) | | | |
| | 1/3.20E−02 | 1.3/3.14E−02 | | | |
| | Gender | | | | |
| | Males | | | | |
| | C: | | | | |
| | (15/199) | | | | |
| | 0.63/4.48E−02 | | | | |
| | Gender-dx | | | | |
| | M-PTSD | | | | |
| | C: | | | | |
| | (3/10) | | | | |
| | 0.86/4.37E−02 | | | | |
| | Gender-dx | | | | |
| | M-SZ | | | | |
| | C: | | | | |
| | (4/42) | | | | |
| | 0.76/4.74E−02 | | | | |
| DYNLL2 | ALL | Gender-dx | Aging | Benzodi- | 15 |
| Dynein | L: | M-SZA | Alcohol | azepines | |
| Light | (5/129) | C: | Alzheimer's | Valproate | |
| Chain | 0.74/3.36E−02 | (23/58) | Disease | | |
| LC8- | | 1.49/4.09E−02 | Metham- | | |
| Type 2 | | | phetamine | | |
| | | | Stress | | |
| | | | SZ | | |
| Hs.550187 | Gender | ALL | Suicide | N/A | 15 |
| | Females | C: | | | |
| | C: | (70/318) | | | |
| | (3/37) | 1.19/3.48E−02 | | | |
| | 0.8/4.23E−02 | Gender-dx | | | |
| | Gender-dx | M- | | | |
| | F-SZA | PSYCHOSIS | | | |
| | C: | C: | | | |
| | (2/6) | (37/121) | | | |
| | 1/3.20E−02 | 1.23/2.54E−02 | | | |
| | M- | M-SZ | | | |
| | PSYCHOSIS | C: | | | |
| | C: | (14/63) | | | |
| | (7/74) | 1.27/3.90E−02 | | | |
| | 0.71/3.44E−02 | | | | |
| | M-SZA | | | | |
| | C: | | | | |
| | (3/32) | | | | |
| | 0.9/1.29E−02 | | | | |
| NRG1 | | ALL | Aging | Ketamine | 15 |
| Neuregulin | | C: | Alcohol | Schizo- | |
| 1 | | (70/318) | Alzheimer's | phrenia | |
| | | 1.2/3.99E−02 | BP | Antipsy- | |
| | | Gender | Chronic | chotics | |
| | | Males | Stress | Valproate | |
| | | C: | Cocaine | | |
| | | (59/273) | COVID-19 | | |
| | | 1.27/1.37E−02 | Depression | | |
| | | Gender-dx | Longevity | | |
| | | M-BP | MDD | | |
| | | C: | Memory | | |
| | | (13/96) | Metham- | | |
| | | 1.66/1.80E−02 | phetamine | | |
| | | M- | Psychosis | | |
| | | PSYCHOSIS | PTSD | | |
| | | C: | Suicide | | |
| | | (37/121) | SZ | | |
| | | 1.25/4.26E−02 | | | |
| | | M-SZA | | | |
| | | C: (23/58) | | | |
| | | 1.32/4.07E−02 | | | |
| TFRC | Gender-dx | ALL | Aging | Valproate | 15 |
| Trans- | M-PTSD | C: | Alcohol | | |
| ferrin | C: | (70/318) | ALZ | | |
| Receptor | (3/10) | 1.29/2.21E−02 | BP | | |
| | 0.9/2.64E−02 | Gender | Cocaine | | |
| | | Males | Depression | | |
| | | C: | Longevity | | |
| | | (59/273) | MDD | | |
| | | 1.35/1.46E−02 | (Recurrent) | | |
| | | Gender-dx | Neuro- | | |
| | | M-SZA | pathic | | |
| | | C: | Pain | | |
| | | (23/58) | SZ | | |
| | | 1.54/3.73E−02 | | | |

TABLE 3
A, and B. Biology of Anxiety Biomarkers. Top CFE3 => 8 (n = 95 probesets, 82 genes). A. Pathway Analyses B. Diseases

| | DAVID GO Functional Annotation | | | | | | | | | | | |
|---|---|---|---|---|---|---|---|---|---|---|---|---|
| | Biological Processes | | | | | KEGG Pathways | | | | Ingenuity Pathways | | |
| | | | | | P- | | | | P- | Top Canonical | | |
| A. | # | Term | Count | % | Value | Term | Count | % | Value | Pathways | P-Value | Overlap |
| Top | 1 | response to drugs | 11 | 13.6 | 1.10E−06 | Hippo | 6 | 7.4 | 1.70E−03 | G-Protein | 5.06E−06 | 2.9% |
| candidate | | | | | | signaling | | | | Coupled | | 8/276 |
| biomarkers | | | | | | pathway | | | | Receptor | | |
| (n = 95 | | | | | | | | | | Signaling | | |
| probesets, | 2 | chemical synaptic | 9 | 11.1 | 1.30E−05 | cAMP | 6 | 7.4 | 5.40E−03 | CREB | 6.87E−06 | 1.8% |
| 82 | | transmission | | | | signaling | | | | Signaling in | | 11/602 |
| genes) | | | | | | pathway | | | | Neurons | | |
| | 3 | response to | 4 | 4.9 | 1.20E−03 | Proteoglycans | 6 | 7.4 | 5.70E−03 | Relaxin | 1.51E−05 | 3.9% |
| | | cAMP | | | | in cancer | | | | Signaling | | 6/153 |
| | 4 | positive | 9 | 11.1 | 1.20E−03 | Pathways in | 8 | 9.9 | 6.80E−03 | Cardiac | 1.61E−05 | 1.9% |
| | | regulation of cell | | | | cancer | | | | Hypertrophy | | 10/538 |
| | | proliferation | | | | | | | | Signaling | | |
| | | | | | | | | | | (Enhanced) | | |
| | 5 | cellular response | 4 | 4.9 | 3.90E−03 | Rap1 | 6 | 7.4 | 7.00E−03 | Molecular | 2.28E−05 | 2.0% |
| | | to retinoic acid | | | | signaling | | | | Mechanisms of | | 9/445 |
| | | | | | | pathway | | | | Cancer | | |

| | David | | | | | Ingenuity Pathways Disease | | |
|---|---|---|---|---|---|---|---|---|
| B. | # | Term | Count | % | P-Value | Diseases and Disorders | P-Value | # Molecules |
| Top | 1 | Psychiatric Disorders | 14 | 17.3 | 5.80E−09 | Neurological Disease | 3.96E−04-1.65E−11 | 67 |
| candidate | 2 | Depression | 11 | 13.6 | 1.00E−08 | Cancer | 3.96E−04-2.96E−09 | 80 |
| biomarkers | 3 | Alcohol consumption | 10 | 12.3 | 3.00E−08 | Gastrointestinal Disease | 3.62E−04-2.96E−09 | 77 |
| (n = 95 | 4 | Attention deficit disorder | 7 | 8.6 | 6.40E−07 | Organismal Injury and | 3.96E−04-2.96E−09 | 80 |
| probesets, | | conduct disorder | | | | Abnormalities | | |
| 82 | | oppositional defiant | | | | | | |
| genes) | | disorder | | | | | | |
| | 5 | Bulimia | 9 | 11.1 | 6.00E−06 | Developmental Disorder | 2.33E−04-4.90E−09 | 24 |
| | 6 | Panic Disorder | 6 | 7.4 | 8.50E−06 | | | |
| | 7 | Tobacco Use Disorder | 34 | 42 | 1.10E−05 | | | |
| | 8 | Tourette Syndrome | 5 | 6.2 | 1.20E−05 | | | |
| | 9 | Breast Cancer | 14 | 17.3 | 1.60E−05 | | | |
| | 10 | Autism | 11 | 13.6 | 2.00E−05 | | | |

TABLE 4
Therapeutics: Drug repurposing for Anxiety using Connectivity
Map⁵ (CMAP) For Top Biomarkers from Table 2 (n = 19).
Direction of expression in high anxiety. 2 out of 4 Decreased
and 10 out of 15 Increased probesets were present in HG-U133A,
the array used for CMAP (from genes ADRA2A, ATP1B2, CCKBR,
FZD10, GAD1, GPX7, GRK4, NRG1, NTRK3, SLC6A2, TFRC). Drugs that
have opposite gene expression effects to the gene expression signature.
Connectivity Map (CMAP) analyses

| rank | cmap name | score | Roles ( |
|---|---|---|---|
| 1 | thalidomide | −1 | Originally introduced as a non-barbiturate |
| | | | hypnotic, but withdrawn from the market due to |
| | | | teratogenic effects. It has been reintroduced and |
| | | | used for a number of immunological and |
| | | | inflammatory disorders. |
| 2 | ethoxyquin | −0.984 | Ethoxyquin is a genotoxic quinoline. |
| 3 | estradiol | −0.979 | Female sex hormone |
| 4 | tetracaine | −0.96 | Local anesthetic |
| 5 | pirenperone | −0.957 | 5-HT2A receptor antagonist described as an |
| | | | antipsychotic and tranquilizer which was never |
| | | | marketed. |
| 6 | atropine | −0.953 | Muscarinic antagonist |
| 7 | 15(S)-15- | −0.933 | Labor induction |
| | methylprostaglandin E2 | | |
| 8 | loperamide | −0.919 | Peripheral opioid receptor agonist used to treat |
| | | | diarrhea |
| 9 | tropicamide | −0.918 | Muscarinic antagonist |
| 10 | esculetin | −0.917 | Plant toxin |
| 11 | isocorydine | −0.916 | Alkaloid |
| 12 | disopyramide | −0.913 | Antiarrhythmic |

TABLE 5
Some predictive biomarkers for anxiety determined
from cross-sectional and longitudinal studies.

| | | | | AUC(O.R. | |
|---|---|---|---|---|---|
| | In | | Best | for All | |
| Predicting | Population | Methodology | Biomarker | Future) | p-value |
| State- High | All | Cross- | ERCC6L2 | 0.681115 | 0.003691 |
| Anxiety | | sectional | | | |
| | | Longitudinal | ERCC6L2 | 0.690086 | 0.033311 |
| | Males | Cross- | ERCC6L2 | 0.717557 | 0.002824 |
| | | sectional | | | |
| | | Longitudinal | DYNLL2 | 0.778926 | 0.027887 |
| | Females | Cross- | PHF21A | 0.778313 | 0.018677 |
| | | sectional | | | |
| | | Longitudinal | PHF21A | 0.901042 | 0.004094 |
| State- High | All | Cross- | SLC6A2 | 0.63173 | 0.002373 |
| Clinical Anxiety | | sectional | | | |
| | | Longitudinal | PTPRD | 0.6903 | 0.001837 |
| | Males | Cross- | ANKRD28 | 0.717962 | 0.000131 |
| | | sectional | | | |
| | | Longitudinal | BRINP2 | 0.706938 | 0.010243 |
| | Females | Cross- | SLC6A2 | 0.758906 | 0.000478 |
| | | sectional | | | |
| | | Longitudinal | TGFB2 | 0.769048 | 0.00435 |
| Trait-First Year | All | Cross- | FZD10 | 0.672018 | 0.007661 |
| Hospitalizations | | sectional | | | |
| | | Longitudinal | SLC15A2 | 0.770313 | 0.020358 |
| | Males | Cross- | ACP1 | 0.698551 | 0.005308 |
| | | sectional | | | |
| | | Longitudinal | FZD10 | 0.75 | 0.045167 |
| | Females | Cross- | 240253_at | 0.803922 | 0.042274 |
| | | sectional | | | |
| | | Longitudinal | NA | | |
| Trait- All | All | Cross- | RPL14 | 1.6929217 | 7.72901E−05 |
| Future Years | | sectional | | | |
| Hospitalizations | | | | | |
| | | Longitudinal | PVALB | 1.759395 | 0.000886 |
| | Males | Cross- | RPL14 | 1.6708136 | 0.000320938 |
| | | sectional | | | |
| | | Longitudinal | SLC6A2 | 1.593965 | 0.013816 |
| | Females | Cross- | TRIM9 | 1.9784747 | 0.018869236 |
| | | sectional | | | |
| | | Longitudinal | PVALB | 3.318625 | 0.001526 |

TABLE 6
Pharmacogenomics. Top biomarkers (from Table 2) that are targets of existing drugs and are changed in expression in opposite direction
to high anxiety. (I)—increased in expression, (D)—decreased in expression. DE—differential expression, AP—Absent/Present.

| | | | Step | | CFE | | | | | |
|---|---|---|---|---|---|---|---|---|---|---|
| | | | 2 | | Poly- | | | | | |
| | | | Priori- | | evidence | | | | | |
| | | | tization | Step 3 | Score for | | | | | |
| | | Step 1 | Total | Validation | Involve- | | | | | |
| | | Discovery | CFG | Anova | ment in | | | | | |
| | | (Change) | Score | p- | Anxiety | Omega-3 | | Mood | | |
| Gene Symbol/ | Probe- | Method/Score | For | value | (Based on | Fatty | Antide- | Stabil- | Benzodi- | Other |
| Gene Name | set | 6 pts | Anxiety | 6 pts | Steps 1-4) | Acids | pressants | izers | azapines | Treatments |
| GAD1 | 205278_{—} | (I) | 11 | 6.79E−01/0 | 22 | (D) | | (D) | | |
| Glutamate | at | DE/4 | | Not | | Lympho- | | neuro- | | |
| Decarboxy- | | 62.3% | | Stepwise | | cytes | | blastoma | | |
| lase 1 | | | | | | Omega-3 | | cells | | |
| | | | | | | fatty | | Lithium, | | |
| | | | | | | acids²⁵⁵ | | Valpro- | | |
| | | | | | | | | ate²⁵⁶ | | |
| | | | | | | | | (D) | | |
| | | | | | | | | Embryonic | | |
| | | | | | | | | Stem | | |
| | | | | | | | | Cell | | |
| | | | | | | | | Carbamaz- | | |
| | | | | | | | | epine²⁵⁷ | | |
| NTRK3 | 215025_{—} | (I) | 4 | 6.58E−01/2 | 19 | | | | | (D) |
| Neurotrophic | at | DE/2 | | Stepwise | | | | | | VT |
| Receptor | | 35.1% | | | | | | | | Clozapine¹²¹ |
| Tyrosine | | | | | | | | | | |
| Kinase 3 | | | | | | | | | | |
| ADRA2A | 209869_{—} | (I) | 4 | 9.07E−01/0 | 17 | | (D) | (D) | | (D) |
| Adreno- | at | AP/4 | | Not | | | larvae | Embryonic | | Clozapine¹²¹ |
| ceptor | | 50% | | Stepwise | | | Norflu- | Stem | | |
| Alpha 2A | | | | | | | oxetine | Cell | | |
| | | | | | | | (SSRI)²⁶⁰ | Valpro- | | |
| | | | | | | | | ate²⁵⁷ | | |
| | | | | | | | | (D) | | |
| | | | | | | | | Embryonic | | |
| | | | | | | | | Stem | | |
| | | | | | | | | Cell | | |
| | | | | | | | | Carbamaz- | | |
| | | | | | | | | epine²⁵⁷ | | |
| FZD10 | 219764_{—} | (I) | 2 | 3.57E−01/2 | 17 | | (D) | | | (D) |
| Frizzled | at | DE/4 | | Stepwise | | | Cell | | | NT2 |
| Class | | 53.2% | | | | | Line | | | human |
| Receptor 10 | | | | | | | Fluox- | | | teratocar- |
| | | | | | | | etine²⁶⁴ | | | cinoma |
| | | | | | | | | | | cells |
| | | | | | | | | | | Cell |
| | | | | | | | | | | Culture |
| | | | | | | | | | | Aripipra- |
| | | | | | | | | | | zole²⁶⁵ |
| | | | | | | | | | | (D) |
| | | | | | | | | | | Hippocampus |
| | | | | | | | | | | (CA1) |
| | | | | | | | | | | Gamma |
| | | | | | | | | | | frequency²⁶⁶ |
| ATP1B2 | 204311_{—} | (I) | 4 | 1.65E−01/2 | 16 | | | (D) | | (D) |
| ATPase | at | DE/2 | | Stepwise | | | | AMY | | straiatum |
| Na+/K+ | | 39% | | | | | | Valpro- | | Haloperi- |
| Trans- | | | | | | | | ate²⁰⁶ | | dol²⁶² |
| porting | | | | | | | | (D) | | (D) |
| Subunit | | | | | | | | Lithium²⁶¹ | | VT |
| Beta 2 | | | | | | | | | | Clozapine¹²¹ |
| | | | | | | | | | | (D) |
| | | | | | | | | | | Prefrontal |
| | | | | | | | | | | cortex |
| | | | | | | | | | | Digoxin²⁶³ |
| CLIC6 | 242913_{—} | (I) | 2 | 5.06E−01/0 | 16 | (D) | | | | (D) |
| Chloride | at | AP/6 | | Not | | Lympho- | | | | AMY |
| Intra- | | 84.2% | | Stepwise | | cytes | | | | Clozapine¹²¹ |
| cellular | | | | | | (females) | | | | (D) |
| Channel 6 | | | | | | Omega-3 | | | | Hippocampus |
| | | | | | | fatty | | | | (CA1) |
| | | | | | | acids²⁵⁵ | | | | Gamma |
| | | | | | | | | | | frequency²⁶⁶ |
| EFNA5 | 1559360_{—} | (I) | 2 | 2.02E−04/4 | 16 | (D) | | | | (D) |
| Ephrin A5 | at | DE/6 | | Nominal | | Brain | | | | Hippocampus |
| | | 80.5% | | | | Omega-3 | | | | (CA1) |
| | | (I) | | | | fatty | | | | Gamma |
| | | AP/4 | | | | acids²⁶⁷ | | | | frequency²⁶⁶ |
| | | 51.3% | | | | | | | | |
| GPX7 | 213170_{—} | (D) | 2 | 4.64E−01/2 | 16 | | (I) | | | (I) |
| Glutathione | at | DE/4 | | Stepwise | | | C. | | | PFC |
| Peroxidase 7 | | 64.3% | | | | | elegans | | | SAM¹⁴⁶ |
| | | | | | | | Mian- | | | |
| | | | | | | | serin²⁵⁸ | | | |
| NTRK3 | 215311_{—} | (I) | 4 | 3.26E−01/2 | 16 | | | | | (D) |
| Neurotrophic | at | DE/4 | | Stepwise | | | | | | VT |
| Receptor | | 53.2% | | | | | | | | Clozapine¹²¹ |
| Tyrosine | | | | | | | | | | |
| Kinase 3 | | | | | | | | | | |
| SLC6A2 | 217214_{—} | (I) | 4 | 3.40E−01/2 | 16 | | (D) | | | |
| Solute | s_at | DE/2 | | Stepwise | | | Cortex | | | |
| Carrier | | 40.3% | | | | | Fluox- | | | |
| Family 6 | | | | | | | etine²⁵⁹ | | | |
| Member 2 | | | | | | | (D) | | | |
| | | | | | | | larvae | | | |
| | | | | | | | Norflu- | | | |
| | | | | | | | oxetine | | | |
| | | | | | | | (SSRI)²⁶⁰ | | | |
| SLC6A4 | 242009_{—} | (I) | 10 | 8.51E−01/0NS | 16 | (D) | (D) | | | (D) |
| Solute | at | AP/2 | | | | Lympho- | Neural | | | Hippocampus |
| Carrier | | 35.1% | | | | cytes | progeni- | | | Oxycodone²⁷⁵ |
| Family 6 | | | | | | Omega-3 | tor cells | | | |
| Member 4 | | | | | | fatty | imipra- | | | |
| | | | | | | acids²⁵⁵ | mine, | | | |
| | | | | | | | citalo- | | | |
| | | | | | | | pram²⁷² | | | |
| | | | | | | | (D) | | | |
| | | | | | | | Blood | | | |
| | | | | | | | Vortiox- | | | |
| | | | | | | | etine²⁷³ | | | |
| | | | | | | | (D) | | | |
| | | | | | | | Blood | | | |
| | | | | | | | Agomela- | | | |
| | | | | | | | tine¹⁸¹ | | | |
| | | | | | | | (D) | | | |
| | | | | | | | DR | | | |
| | | | | | | | Sertra- | | | |
| | | | | | | | line²⁷⁴ | | | |
| TMEM138 | 223113_{—} | (D) | 4 | 8.63E−01/0 | 16 | | | | | |
| Trans- | at | DE/4 | | Not | | | | | | |
| membrane | | 63% | | Stepwise | | | | | | |
| Protein 138 | | | | | | | | | | |
| ANKRD28 | 229307_{—} | (I) | 0 | 6.20E−01/2 | 15 | | | | | |
| Ankyrin | at | DE/6 | | Stepwise | | | | | | |
| Repeat | | 80.5% | | | | | | | | |
| Domain 28 | | | | | | | | | | |
| CCKBR | 210381_{—} | (I) | 4 | 4.37E−01/2 | 15 | | | | | (D) |
| Cholecysto- | s_at | DE/2 | | Stepwise | | | | | | VT |
| kinin | | 48% | | | | | | | | Clozapine¹²¹ |
| B Receptor | | | | | | | | | | |
| DYNLL2 | 229106_{—} | (D) | 4 | 1.96E−01/2 | 15 | | | (I) | (I) | |
| Dynein Light | at | DE/2 | | Stepwise | | | | Embryonic | AMY | |
| Chain | | 39.3% | | | | | | stem | Benzodi- | |
| LC8-Type 2 | | | | | | | | cell | azepines²⁸ | |
| | | | | | | | | Valpro- | | |
| | | | | | | | | ate²⁵⁷ | | |
| GRK4 | 210600_{—} | (I) | 0.00 | 3.40E−01/2 | 15 | | | | | (D) |
| G Protein- | s_at | DE/6 | | Stepwise | | | | | | Blood |
| Coupled | | 81.8% | | | | | | | | Clozapine²⁶⁸ |
| Receptor | | | | | | | | | | (D) |
| Kinase 4 | | | | | | | | | | Hippocampus |
| | | | | | | | | | | (CA1) |
| | | | | | | | | | | Gamma |
| | | | | | | | | | | frequency²⁶⁶ |
| Hs.550187 | 240253_{—} | (I) | 0 | 2.74E−01/2 | 15 | | | | | |
| | at | DE/6 | | Stepwise | | | | | | |
| | | 88.3% | | | | | | | | |
| NRG1 | 208232_{—} | (I) | 4 | 6.66E−01/2 | 15 | | (D) | (D) | | (D) |
| Neuregulin 1 | x_at | DE/4 | | Stepwise | | | Hippo- | neuro- | | blood |
| | | 65.8% | | | | | campus, | blastoma | | Antipsy- |
| | | | | | | | PFC | cells | | chotic |
| | | | | | | | Keta- | Lithium | | response |
| | | | | | | | mine²⁶⁹ | Resis- | | in |
| | | | | | | | | tance²⁷⁰ | | men²⁷¹ |
| | | | | | | | | (D) | | |
| | | | | | | | | neuro- | | |
| | | | | | | | | blastoma | | |
| | | | | | | | | cells | | |
| | | | | | | | | Valpro- | | |
| | | | | | | | | ate²⁵⁶ | | |
| TFRC | 207332_{—} | (D) | 2 | 7.11E−01/2 | 15 | (I) | (I) | (I) | | (I) |
| Transferrin | s_at | DE/4 | | Stepwise | | PFC | Dentate | NT2.D1 | | Clozapine²⁷⁸ |
| Receptor | | 56% | | | | (males) | gyrus | cells | | (I) |
| | | | | | | Omega-3 | Fluox- | Valpro- | | Mucosal |
| | | | | | | fatty | etine²⁷⁶ | ate²⁷⁷ | | Biopsy |
| | | | | | | acids²⁵⁵ | (I) | | | Rofecoxib²⁷⁹ |
| | | | | | | (I) | basal | | | (I) |
| | | | | | | Lympho- | forebrain | | | HCAEC |
| | | | | | | cytes | TCA⁴³ | | | Berberine²⁸⁰ |
| | | | | | | Omega-3 | | | | (I) |
| | | | | | | fatty | | | | Whole |
| | | | | | | acids²⁵⁵ | | | | Brains |
| | | | | | | | | | | Methylphe- |
| | | | | | | | | | | nidate²⁸¹ |

### Discussion

We describe a novel and comprehensive effort to discover and validate blood biomarkers of relevance to anxiety, including testing them in independent cohorts to evaluate predictive ability and clinical utility. These biomarkers also open a window into understanding the biology of anxiety disorders, as well as indicate new and more precise therapeutic approaches. Biomarkers may be useful for matching patients to medications and measuring response to treatment (pharmacogenomics) (FIG. 4, Tables 2 and 6), as well as new drug discovery and repurposing (Table 4).

Overall, this work is a major step forward towards understanding, diagnosing and treating anxiety disorders. We hope that our trait biomarkers for future risk may be useful in preventive approaches before the full-blown disorder manifests itself (or re-occurs). Prevention could be accomplished with social, psychological, or biological interventions (i.e. early targeted use of medications or nutraceuticals). Given the fact that 1 in 5 people will have a clinical anxiety disorders episode in their lifetime, that anxiety disorders can severely affect quality of life, sometimes leading to suicides, and that not all patients respond to current treatments or the treatments are addictive, the need for and importance of efforts such as ours cannot be overstated.

1. A therapeutic compound, selected from one or more compounds from Tables 4 and Table 6, in a method of mitigating and anxiety disorder in a subject in need thereof for example wherein the therapy is a drug, a natural compound, and combinations thereof.

2. The therapeutic compound, for use according to claim 1, wherein the drug is estradiol, loperamide, omage-3 fatty acids, lithium, fluoxetine, norfluoxetine, imipramine, citalopram, carbamazepine, aripiprazole, haloperidol, mianserin, SAM, vortioxetine, agomelatine, sertraline, benzodiazepine, clozapine, ketamine, valproate, clozapine, rofecoxib, berberine, methylphenidate, or berberine.

3. The therapeutic compound, for use according to claim 1, wherein the subject has anxiety disorder.

4. The therapeutic compound, for use according to claim 1, wherein the subject is a male subject or a female subject.

5. A computer-implemented method for assessing the level of anxiety in a subject, and for assessing risk of developing anxiety disorder in the future in a subject, the method comprising: computing a score based on RNA level, protein level, DNA methylation, a single nucleotide polymorphism, for a panel of biomarkers chosen from Table 2, Table 5, and FIG. 2, in a sample obtained from a subject; computing a score based on a reference expression level of the panel of biomarkers; and identifying a difference between the score in the sample obtained from the subject and the score in the reference sample, wherein the difference in the score in the sample obtained from the subject and the score in the reference sample indicates an anxiety disorder, and a risk of future anxiety disorders.

6. The computer implemented method according to claim 5, wherein the subject is:
a male or a female and the biomarker is at least one biomarker selected from the group consisting of: ERCC6L2, SLC6A2, FZD10, and RPL14; or
a female and the biomarker is at least one biomarker selected from the group consisting of: PHF21A, SLC6A2, 240253_at, and TRIM9; or
a male and the biomarker is at least one biomarker selected from the group consisting of: ERCC6L2, ANKRD28, ACP1, and RP

7. The methods of claims 5 and 6, further including the steps of:
identifying a difference between the score in the sample obtained from the subject and the score in the reference sample,
administering treatment to the subject, wherein the treatment reduces the difference between the score in the sample from the subject and the score in the reference sample, and mitigates anxiety in the subject, or the risk for developing anxiety disorder in the subject.

8. The method of claim 7, further including, the step of:
measuring a change in the score after treatment.

9. The method of claim 8, wherein the treatment is at least one treatment selected from the group consisting of: lifestyle modification, electro-magnetic intervention, and administering a therapeutic compound.

10. The method of claim 9, wherein the therapy is selected by a computer-implemented method selected from the group consisting of one or more psychiatric compounds from Table 6, and wherein each therapy selection is based on a panel of one or more individual biomarkers.

11. The method of claim 7, wherein the therapy is selected based on a panel of individual biomarkers changed in a subject, by a computer-implemented method for therapy selection, and comprises, of one or more compounds in Table 4.

12. A method for assessing and mitigating anxiety disorder, comprising determining an expression level of a panel of biomarkers listed in Table 2, Table 5 and FIG. 2, in a sample for example wherein the sample comprises a peripheral tissue, blood, saliva, cerebrospinal fluid (CSF), serum, urine, or stool; wherein the expression level of the biomarkers in the sample is different relative to a reference expression level; and includes the steps of:
identifying the subject currently having or at risk of having an anxiety disorder based on a biomarker panel score relative to a biomarker panel score of a reference panel;
selecting a therapy based on the score from the biomarkers; and
administering one or more compounds selected from the group consisting of compounds selected from the groups consisting of compounds selected from Tables 4 and Table 6.

13. A composition comprising one or more compounds from Tables 4 and Table 6, for use in a method for treating anxiety disorder.
